(19) United States
(12) Patent Application Publication
(10) Pub. No.: US 2026/0224787 A1
(43) Pub. Date: Aug. 6, 2026

(54) Breast Pump, Breast Pumping Apparatus and Horn Cover Thereof

(71) Applicant: NINGBO XIAOWU BRAND MANAGEMENT CO., LTD., Ningbo (CN)

(72) Inventors: Qiansheng Ye, Ningbo (CN); Yongliang Wang, Ningbo (CN); Jinwei Xin, Ningbo (CN); Lingfeng Zhao, Ningbo (CN)

(21) Appl. No.: 19/575,812

(22) Filed: Mar. 23, 2026

Related U.S. Application Data
(63) Continuation of application No. PCT/CN2025/088376, filed on Apr. 10, 2025.

(30) Foreign Application Priority Data
Nov. 8, 2024 (CN) 202422727930.9
Jan. 13, 2025 (CN) 202510051358.5
Jan. 13, 2025 (CN) 202520073439.0
Jan. 13, 2025 (CN) 202520073458.3
Apr. 10, 2025 (WO) PCT/CN2025/088376

(51) Int. Cl. A61M 1/06

(52) U.S. Cl. CPC A61M 1/064

(57) ABSTRACT

A breast pumping apparatus includes an integrated device body, a rear cover, and a milk bottle, the device body has a spatially variable milk suction chamber and a milk discharge channel selectively communicated to the milk suction chamber. The rear cover has an air channel and is installed at the rear of the device body, a negative pressure chamber is formed between the device body and the rear cover, the air channel of the rear cover is communicated to the negative pressure chamber. The pressure state of the milk suction chamber and the connectivity state of the milk discharge channel change with the pressure state of the negative pressure chamber.

### CROSS REFERENCE OF RELATED APPLICATION

This application is a Continuation Application of PCT/CN2025/088376, filing date Apr. 10, 2025, which claims priority under 35 U.S.C. 119(a-d) to Chinese application numbers CN202422727930.9, filing date Nov. 08, 2024; CN202510051358.5, filing date Jan. 13,025; CN202520073439.0, filing date Jan. 13, 2025; and CN202520073458.3, filing date Jan. 13, 2025, the afore-mentioned patent applications are hereby incorporated by reference in their entireties.

### BACKGROUND OF THE PRESENT INVENTION

### FIELD OF INVENTION

The present invention relates to the field of maternal and infant products, and more particularly to a breast pump, a breast pumping apparatus and horn cover thereof.

### DESCRIPTION OF RELATED ARTS

In Chinese invention patent application CN118873774A, the inventor has disclosed a breast pump which comprises a device body and a horn cover. The device body has a suction chamber, a milk collection chamber, and a milk discharge channel connecting the milk suction chamber and the milk collection chamber. The horn cover comprises an outer first extension, a rotatable second outer extension, and an inner extension. The outer second extension and the inner extension extend outward from opposite sides of the outer first extension. The inner extension is detachably installed on the device body. When the inner extension is installed on the device body, the horn cover is positioned on the device body. In this conventional breast pump, the inner surface of the inner extension of the horn cover is a complete arc surface. When the user uses the breast pump, the flow direction of the milk temporarily stored in the milk suction chamber lacks effective guidance, causing the milk to flow randomly within the milk suction chamber. Especially when a user is leaning against the back of a sofa or bed while pumping, their posture causes the horn cover to be tilted downwards. This means the height of the end of the inner extension connecting to the outer first extension is lower than the height of the end facing away from the outer first extension, leading to milk backflow. In other words, the existing breast pump does not allow users to pump while leaning against the back of a sofa or bed; it only allows users to pump with their upper body upright or tilted forward to avoid milk backflow. Furthermore, in the existing breast pumping apparatus, the support housing and negative pressure forming part of the negative pressure forming unit, as well as the horn cover, are separate components. That is, the support housing, negative pressure forming part, and horn cover are injection molded separately using different molds and then assembled. Therefore, the separate design of the breast pumping apparatus results in higher costs and lower production efficiency.

In addition, in Chinese invention patent publication number CN118787802A, the inventors have disclosed a self-adhesive breast pumping apparatus which comprises a one-way valve, a self-adhesive cover, a negative pressure forming unit, and a bottle. The negative pressure forming unit has a negative pressure chamber, a gas exchange channel, a milk suction chamber, an assembly opening, and a milk discharge channel. The gas exchange channel is connected to the negative pressure chamber, and the assembly opening and the milk discharge channel are respectively connected to the milk suction chamber. The milk suction chamber and the negative pressure chamber are separated by a deformable component of the negative pressure forming unit. The self-adhesive cover is installed in the assembly opening of the negative pressure forming unit. The one-way valve is disposed in the negative pressure forming unit to selectively open and close the milk discharge channel. The milk bottle is installed in the negative pressure forming unit and covers the one-way valve. The self-adhesive cover comprises a horn-shaped cover body, and there is a movable space between the horn-shaped cover body and the negative pressure forming unit. The movable space allows the horn-shaped cover body to wrap around a part of the breast after flipping and restoring, thereby enabling the self-adhesive breast pumping apparatus to be reliably adsorbed onto the breast. During milk expression, as the amount of milk increases, the weight of the self-adhesive breast pumping apparatus also increases. This increased space causes the horn-shaped cover to deform, resulting in the nipple losing its balance within the milk suction chamber of the negative pressure forming unit. The direction of milk ejection relative to the negative pressure forming unit changes, causing milk to first spray onto the wall of the milk suction chamber and then be reflected between the horn-shaped cover and the areola, failing to enter the milk discharge channel of the negative pressure forming unit. This leads to milk leakage, affecting both the expression efficiency and the suction properties. Furthermore, the deformation of the self-adhesive cover due to the increased space also affects the suction properties between the horn-shaped cover and the breast.

### SUMMARY OF THE PRESENT INVENTION

An object of the present invention is to provide a breast pump, a breast pumping apparatus and its horn cover, wherein the main body of the breast pumping apparatus is integral, which is beneficial to reduce the production cost of the breast pumping apparatus and improve the production efficiency of the breast pumping apparatus.

An object of the present invention is to provide a breast pump, a breast pumping apparatus and its horn cover, wherein when the milk bottle of the breast pumping apparatus is installed with the rear cover, the rim of the milk bottle abuts against the device body. In this way, the device body can provide pre-pressure to the milk bottle to prevent the milk bottle from loosening. At the same time, the device body can prevent gaps from forming between the device body and the rim of the milk bottle, thereby allowing milk to be reliably stored in the milk storage chamber of the milk bottle.

An object of the present invention is to provide a breast pump, a breast pumping apparatus and its horn cover, wherein the surface of the horn cover of the breast pumping apparatus for defining the horn space is a concave arc surface, so that the horn cover can be self-adhesive to the breast to achieve the "naked suction" function.

An object of the present invention is to provide a breast pump, a breast pumping apparatus and a horn cover thereof, wherein the breast pumping apparatus allows the user to suckle to help the user complete the breast pumping in a comfortable posture.

An object of the present invention is to provide a breast pump, a breast pumping apparatus and its horn cover, wherein the horn cover of the breast pumping apparatus provides a guide channel for guiding milk temporarily stored in the milk suction chamber to the milk discharge channel to avoid the problem of milk backflow.

An object of the present invention is to provide a breast pump, a breast pumping apparatus and its horn cover, wherein the guide groove of the horn cover increases the space for temporary storage of milk in the cover part of the horn cover, and for users with a large milk output, the problem of milk backflow caused by the milk level being too high in the milk suction chamber of the horn cover during the pumping process can be avoided.

According to an aspect of the invention, the present invention provides a flexible horn cover having a cover space and comprising an abutting portion located in the cover space, the abutting portion being used to abut against an areola, wherein a surface of the horn cover defining the cover space is a concave arc surface, and a portion of the horn cover defining the cover space is capable of being flipped backward.

According to an embodiment of the present invention, a top of the horn cover is formed with a top protrusion.

According to an embodiment of the present invention, a top of the horn cover is formed with a top protrusion, a bottom thereof is formed with a bottom protrusion, and two opposite lateral sides are respectively formed with contraction portions, wherein a vertical distance between the top protrusion and the abutting portion is greater than a vertical distance between the bottom protrusion and the abutting portion, and the vertical distance between the bottom protrusion and the abutting portion is greater than a vertical distance between each contraction portion and the abutting portion.

According to an embodiment of the present invention, a curvature of a bottom portion of the surface of the horn cover that defines the cover space is greater than a curvature of the top portion thereof.

According to an embodiment of the present invention, the horn cover comprises a support ring that is protruded from an outer surface of the horn cover.

According to an embodiment of the present invention, the horn cover comprises a horn portion and a mounting portion extended rearward from a rear of the horn portion, wherein the cover space of the horn cover is formed in the horn portion, and the abutting portion of the horn cover is formed at a connection position between the horn portion and the mounting portion.

According to an embodiment of the present invention, the mounting portion is extended outward from the rear of the horn portion in a manner that an inner wall of the mounting portion is an inclined inner wall.

According to an embodiment of the present invention, the horn cover comprises a tapered portion disposed on an outer edge of the horn portion, and a thickness of the tapered portion is greater than a thickness of a periphery of an opening of the horn portion.

According to an embodiment of the present invention, the horn cover comprises an annular frame disposed on the mounting portion.

According to an embodiment of the present invention, a hardness of the mounting portion is greater than a hardness of the horn portion.

According to an embodiment of the present invention, the horn cover has a positioning boss or a positioning groove.

According to another aspect, the present invention provides a breast pumping apparatus which comprises a device body which is an integral piece, a rear cover and a milk bottle, and has a negative pressure chamber, wherein the device body comprises:

a cylindrical body;

a deformable portion which is integrally formed at a rear of the cylinder, and the device body has a milk suction chamber defined between the cylindrical body and the deformable portion;

an abutment portion which is integrally formed at a bottom of the cylindrical body, and the device body has a milk discharge channel which is extended from an inner wall of the cylindrical body to an abutment surface of the abutment portion; and a horn cover having a cover space and comprising an abutment portion located in the cover space, the abutment portion being used to abut against an areola, wherein a surface of the horn cover defining the cover space is a concave arc surface, and a portion of the horn cover defining the cover space is capable of being flipped backward, wherein the horn cover is integrally formed on a front of a cylindrical body, wherein the rear cover is installed on the cylindrical body, a negative pressure chamber is formed between the rear cover and the deformable portion, wherein the milk bottle is detachably installed on the rear cover, wherein when the milk bottle is installed on the rear cover, a mouth of the milk bottle abuts against the abutment surface of the abutment portion, and the milk discharge channel is capable of connecting the milk suction chamber and a milk storage chamber of the milk bottle.

According to an embodiment of the present invention, an inner wall of the device body for forming the milk suction chamber is inclinedly extended along a direction from a connection position between the horn cover and the cylindrical body towards the deformable portion.

According to an embodiment of the present invention, the device body comprises a panel portion integrally formed on the cylindrical body and surrounding a middle of the cylindrical body, wherein an edge of the rear cover is mounted on an edge of the panel portion.

According to an embodiment of the present invention, the rear cover comprises a cover shell, an inner abutment ring arm, and an assembly ring, wherein the inner abutment ring arm is extended integrally from a middle of the cover shell, and the assembly ring is integrally formed at a bottom of the cover shell, wherein the inner abutment ring arm sealably abuts against the cylindrical body to form the negative pressure chamber between the rear cover and the deformable portion, the assembly ring is located below the abutment portion, and the milk discharge channel corresponds to a ring channel of the assembly ring, an edge of the cover shell is mounted to the edge of the panel portion, the mouth of the milk bottle passes through the ring channel of the assembly ring and is mounted on the assembly ring, and the mouth of the milk bottle abuts against the abutment surface of the abutment portion.

According to an embodiment of the present invention, the rear cover comprises an outer abutment ring arm that is integrally extended from the middle of the cover shell and is located outside the inner abutment ring arm, wherein the outer abutment ring arm is mounted on an inner side of the panel portion.

According to an embodiment of the present invention, the breast pumping apparatus comprises a duckbill valve which is disposed on the device body and is used to control connectivity of the milk discharge channel.

According to an embodiment of the present invention, the duckbill valve is installed on the abutment portion of the device body.

According to an embodiment of the present invention, the duckbill valve and the device body are integrally formed.

According to an embodiment of the present invention, the device body and the duckbill valve are integrally made of silicone material.

According to an embodiment of the present invention, the abutment portion has a vent hole extended from the abutment surface to a side of the abutment portion opposite to the abutment surface.

According to an embodiment of the present invention, the abutment portion has an exhaust groove being disposed on the side of the abutment portion opposite to the abutment surface, and the exhaust groove is extended from the vent hole to an edge of the abutment portion.

According to an embodiment of the present invention, the rear cover has an exhaust groove which is communicated with the vent hole of the abutment portion.

According to an embodiment of the present invention, the abutment portion has an exhaust groove disposed on the abutment surface of the abutment portion, and extended from an edge of the abutment portion toward the milk discharge channel.

According to an embodiment of the present invention, the device body comprises an annular frame disposed on the cylindrical body to maintain a shape of the cylindrical body.

According to another aspect, the present invention provides a deformable horn cover comprising a self-adhesive cover and a support ring protruded from an outer wall of the self-adhesive cover, wherein the self-adhesive cover and the support ring are integral.

According to an embodiment of the present invention, the self-adhesive cover comprises a shoulder ring, a mounting ring, and a horn cover element which is deformable, wherein the mounting ring is extended from an inner edge of the shoulder ring to one side, and the horn cover element is extended from the outer edge of the shoulder ring in the opposite direction, wherein the support ring is disposed at a connection position between the shoulder ring and the horn cover element, and the support ring surrounds the shoulder ring.

According to an embodiment of the present invention, the self-adhesive cover comprises a blocking portion, the blocking portion further including an installing ring and a deformable blocking ring, the blocking ring is extended inwardly from one end of the installing ring, wherein the installing ring is detachably mounted to the mounting ring.

According to an embodiment of the present invention, the self-adhesive cover comprises a deformable blocking ring extended integrally inward from a connection position between the shoulder ring and the mounting ring.

According to an embodiment of the present invention, the horn cover comprises a tapered portion disposed at a periphery of the self-adhesive cover at an external opening of the self-adhesive cover, and a thickness of the tapered portion is greater than a thickness of the self-adhesive cover at the periphery of the external opening.

According to an embodiment of the present invention, a thickness of the portion of the self-adhesive cover located outside the support ring gradually decreases from the support ring outwards.

According to an embodiment of the present invention, a curvature of a lower inner wall of the horn cover is greater than a curvature of an upper inner wall.

According to an embodiment of the present invention, the self-adhesive cover comprises two annular frames, one annular frame is disposed on the mounting ring to maintain a shape of the mounting ring, and the other assembly ring body is disposed on the installing ring to maintain a shape of the installing ring.

According to an embodiment of the present invention, the self-adhesive cover comprises an annular frame disposed on the mounting ring to maintain a shape of the mounting ring.

According to an embodiment of the present invention, the self-adhesive cover comprises an annular frame disposed on the mounting ring.

According to another aspect, the present invention provides a breast pumping apparatus, comprising:

a device body having a milk suction chamber, and a milk discharge channel and an assembly opening respectively communicated with the milk suction chamber; and a horn cover comprising a self-adhesive cover and a support ring protruded from an outer wall of the self-adhesive cover, wherein the self-adhesive cover and the support ring are integral, wherein the horn cover is installed on the device body at the assembly opening of the device body, forming an active space between the device body and the self-adhesive cover, and the support ring is located in the active space to abut against the device body.

According to another aspect, the present invention provides a breast pumping apparatus, comprising:

a three-way component having a front opening, a rear opening and a bottom opening;

a device body comprising a cover portion and a deformable portion, and has a milk suction chamber, wherein the deformable portion is disposed at a tail end of the cover portion, and the milk suction chamber is formed between the cover portion and the deformable portion, the cover portion has an areola abutment portion, a milk discharge channel, and a guide groove, the areola abutment portion is located in a middle section of the cover portion, the milk discharge channel is communicated with the milk suction chamber at a rear of the cover portion, the guide groove is inclinedly extended from the areola abutment portion to the milk discharge channel, the rear of the cover portion is installed on the three-way component through the front opening of the three-way component, the deformable portion is extended out of the three-way component through the rear opening of the three-way component, the milk discharge channel of the cover portion is communicated with the bottom opening of the three-way component;

a rear cover having an air channel, wherein the rear cover is installed on the three-way component, a periphery of the deformable portion is clamped between the three-way component and the rear cover, the breast pumping apparatus forms a negative pressure chamber between the deformable portion and the rear cover, and the air channel of the rear cover is communicated to the negative pressure chamber;

a duckbill valve which is mounted on a bottom of the three-way component for selectively opening and closing the bottom opening of the three-way component; and

a milk bottle which is mounted at the bottom of the three-way component and the milk bottle covers the duckbill valve.

According to an embodiment of the present invention, the areola abutment portion of the cover body is deformable.

According to an embodiment of the present invention, the cover portion comprises a cover body and an inner liner, the milk discharge channel of the cover portion is formed in the cover body, the areola abutment portion and the guide groove of the cover portion are formed in the inner liner, and the inner liner is detachably installed in the middle section of the cover body, such that the areola abutment portion of the cover portion is located in the middle section of the cover portion.

According to an embodiment of the present invention, the cover body has a body positioning groove, the inner liner has a liner positioning boss, and the inner liner is installed on the cover body by means of inserting the liner positioning boss of the inner liner into the body positioning groove of the cover body.

According to an embodiment of the present invention, a position of the guide groove of the cover portion corresponds to a position of the inner liner positioning boss of the inner liner.

According to an embodiment of the present invention, the inner liner has a notch, the guide groove of the cover portion is extended to the notch, and after the inner liner is installed in the middle section of the cover portion, the milk discharge channel of the cover portion corresponds to the notch of the inner liner.

According to an embodiment of the present invention, an inner wall of the cover body is a concave arc surface.

According to an embodiment of the present invention, a top of the cover body is formed with a top protrusion, a bottom thereof is formed with a bottom protrusion, and two opposite lateral sides are respectively formed with contraction portions, a vertical distance between the top protrusion and the areola abutment portion is greater than a vertical distance between the bottom protrusion and the areola abutment portion, and the vertical distance between the bottom protrusion and the areola abutment portion is greater than a vertical distance between each contraction portion and the areola abutment portion.

According to an embodiment of the present invention, a curvature of the bottom of the cover body is greater than a curvature of the top of the cover body.

According to an embodiment of the present invention, the cover portion comprises a support ring protrudingly disposed on an outer wall of the cover body, and the support ring is protruding toward the three-way component.

According to another aspect, the present invention provides a breast pumping apparatus comprising:

a three-way component having a front opening, a rear opening, and a milk discharge channel;

a device body comprising a cover portion and a deformable portion, the cover portion has an areola abutment portion and a guide channel, the areola abutment portion is located in a middle section of the cover portion, the guide channel is extended from the areola abutment portion to a tail end of the cover portion, the cover portion is installed on the three-way component through the front opening of the three-way component., the guide channel of the cover portion is extended to the milk discharge channel of the three-way component, the deformable portion is installed on the three-way component to close the rear opening of the three-way component, the breast pumping apparatus forms a milk suction chamber in the cover portion, the three-way component, and the deformable portion, the milk suction chamber is communicated to the milk discharge channel of the three-way component;

a rear cover having an air channel, the rear cover is installed on the three-way component, a periphery of the deformable portion is clamped between the three-way component and the rear cover, the breast pumping apparatus forms a negative pressure chamber between the deformable portion and the rear cover, and the air channel of the rear cover is communicated to the negative pressure chamber;

a duckbill valve which is mounted on a bottom of the three-way component for selectively opening and closing the bottom opening of the three-way component; and a milk bottle mounted at the bottom of the three-way component and the milk bottle covers the duckbill valve.

According to an embodiment of the present invention, the areola abutment portion of the cover body is deformable.

According to an embodiment of the present invention, the cover portion comprises a cover body and an inner liner, the milk discharge channel of the cover portion is formed in the cover body, the areola abutment portion and the guide groove of the cover portion are formed in the inner liner, and the inner liner is detachably installed in the middle section of the cover body, such that the areola abutment portion of the cover portion is located in the middle section of the cover portion.

According to an embodiment of the present invention, the cover body has a body positioning groove, the inner liner has a liner positioning boss, and the inner liner is installed on the cover body by means of inserting the liner positioning boss of the inner liner into the body positioning groove of the cover body.

According to an embodiment of the present invention, a position of the guide groove of the cover portion corresponds to a position of the inner liner positioning boss of the inner liner.

According to an embodiment of the present invention, an inner wall of the cover body is a concave arc surface.

According to an embodiment of the present invention, a top of the cover body is formed with a top protrusion, a bottom thereof is formed with a bottom protrusion, and two opposite lateral sides are respectively formed with contraction portions, a vertical distance between the top protrusion and the areola abutment portion is greater than a vertical distance between the bottom protrusion and the areola abutment portion, and the vertical distance between the bottom protrusion and the areola abutment portion is greater than a vertical distance between each contraction portion and the areola abutment portion.

According to an embodiment of the present invention, a curvature of the bottom of the cover body is greater than a curvature of the top of the cover body.

According to an embodiment of the present invention, the cover portion comprises a support ring protrudingly disposed on an outer wall of the cover body, and the support ring is protruding toward the three-way component.

### BRIEF DESCRIPTION OF THE DRAWINGS

FIG. 1 is a perspective view of a breast pump according to a preferred embodiment of the present invention.

FIG. 2 is a perspective view of a breast pumping apparatus of the breast pump according to the above-described preferred embodiment of the present invention.

FIG. 3 is another perspective view of the breast pumping apparatus of the breast pump according to the above-described preferred embodiment of the present invention.

FIG. 4 is a side view of the breast pumping apparatus of the breast pump according to the above-described preferred embodiment of the present invention.

FIG. 5 is an exploded view of the breast pumping apparatus of the breast pump according to the above-described preferred embodiment of the present invention.

FIG. 6 is another exploded view of the breast pumping apparatus of the breast pump according to the above-described preferred embodiment of the present invention.

FIG. 7 is a perspective view of a device body of the breast pumping apparatus of the breast pump according to the above-described preferred embodiment of the present invention.

FIG. 8 is a partial enlarged view of a location in FIG. 7.

FIG. 9 is a perspective view of the device body of the breast pumping apparatus of the breast pump according to the above-described preferred embodiment of the present invention.

FIG. 10 is a partial enlarged view of a location of FIG. 9.

FIG. 11 is a perspective cross-sectional view of the breast pumping apparatus of the breast pump according to the above-described preferred embodiment of the present invention.

FIG. 12 is a magnified view of a partial location in FIG. 11.

FIG. 13 is a cross-sectional view of the breast pumping apparatus of the breast pump according to the above-described preferred embodiment of the present invention from a plan view perspective.

FIG. 14A is a cross-sectional schematic view of a modified example of the breast pumping apparatus of the breast pump according to the above-described preferred embodiment of the present invention.

FIG. 14B is a cross-sectional schematic diagram of another modified example of the breast pumping apparatus of the breast pump according to the above-described preferred embodiment of the present invention.

FIG. 15 is an exploded view of another breast pumping apparatus of the breast pump according to the above-described preferred embodiment of the present invention.

FIG. 16 is another exploded view of the breast pumping apparatus of the breast pump according to the above-described preferred embodiment of the present invention.

FIG. 17A is a cross-sectional schematic view of the breast pumping apparatus of the breast pump according to the above-described preferred embodiment of the present invention.

FIG. 17B is a cross-sectional schematic view of a modified example of the breast pumping apparatus of the breast pump according to the above-described preferred embodiment of the present invention.

FIG. 18 is a perspective view of a breast pump according to a preferred embodiment of the present invention.

FIG. 19 is a three-dimensional cross-sectional view of the breast pump according to the above-described preferred embodiment of the present invention.

FIG. 20 is a cross-sectional view of the breast pump according to the above-described preferred embodiment of the present invention from a plan view perspective.

FIG. 21 is an exploded view of the breast pump according to the above-described preferred embodiment of the present invention.

FIG. 22 is another exploded view of the breast pump according to the above-described preferred embodiment of the present invention.

FIG. 23 is a cross-sectional schematic view of a modified example of the breast pump according to the above-described preferred embodiment of the present invention.

FIG. 24 is a cross-sectional schematic view of the breast pump according to another preferred embodiment of the present invention.

FIG. 25 is an exploded view of the breast pump according to the above-described preferred embodiment of the present invention.

FIG. 26 is another exploded view of the breast pump according to the above-described preferred embodiment of the present invention.

FIG. 27 is a perspective view of a breast pump according to a preferred embodiment of the present invention.

FIG. 28 is a cross-sectional schematic view of the breast pump according to the above-described preferred embodiment of the present invention.

FIG. 29 is an exploded view of the breast pump according to the above-described preferred embodiment of the present invention.

FIG. 30 is another exploded view of the breast pump according to the above-described preferred embodiment of the present invention.

FIG. 31 is a cross-sectional schematic view of a first modified example of the breast pump according to the above-described preferred embodiment of the present invention.

FIG. 32 is a cross-sectional schematic view of a second modified example of the breast pump according to the above-described preferred embodiment of the present invention.

FIG. 33 is a cross-sectional schematic view of a third modified example of the breast pump according to the above-described preferred embodiment of the present invention.

FIG. 34 is a cross-sectional schematic view of a fourth modified example of the breast pump according to the above-described preferred embodiment of the present invention.

### DETAILED DESCRIPTION OF THE PREFERRED EMBODIMENT

Before detailing any embodiment of the invention, it should be understood that the invention, in its application, is not limited to the details of the construction and arrangement of the components set forth in the following description or illustrated in the following figures. The invention can have other embodiments and can be practiced or carried out in various ways. Furthermore, it should be understood that the wording and terminology used herein are for descriptive purposes and should not be considered limiting. The use of "comprising" or "having" and variations thereof is intended to cover the items set forth below and their equivalents, as well as any additional items. Unless otherwise specified or limited, the terms "installation", "connection", "support", and "linkage", and variations thereof are used broadly and cover both direct and indirect installation, connection, support, and linking. Moreover, "connect" and "link" are not limited to physical or mechanical connections or links.

Furthermore, firstly, in the disclosure of the present invention, the terms "longitudinal", "lateral", "upper", "lower", "front", "rear", "left", "right", "vertical", "horizontal", "top", "bottom", "inner", and "outer", etc., indicate the orientation or positional relationship based on the orientation or positional relationship shown in the accompanying drawings. They are only for the convenience of describing the present invention and simplifying the description, and do not indicate or imply that the device or element referred to must have a specific orientation, or be constructed and operated in a specific orientation. Therefore, the above terms should not be construed as limiting the invention. Secondly, the term "a" or "an" should be understood as "at least one" or "one or more", that is, in one embodiment, the number of an element can be one, while in another embodiment, the number of the element can be multiple. The term "a" or "an" should not be construed as a limitation on the quantity.

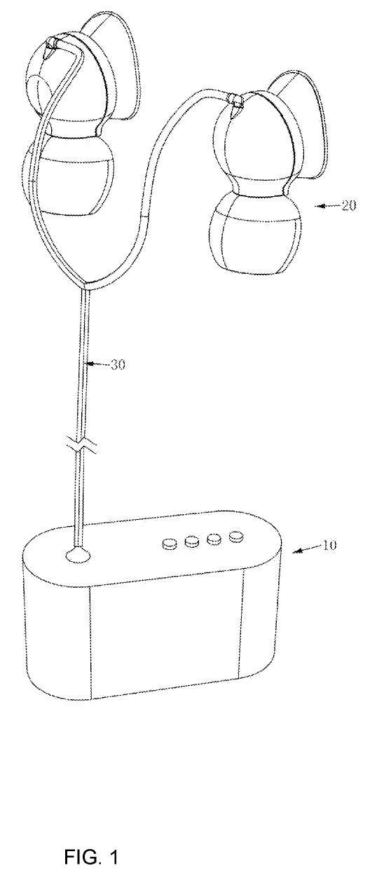

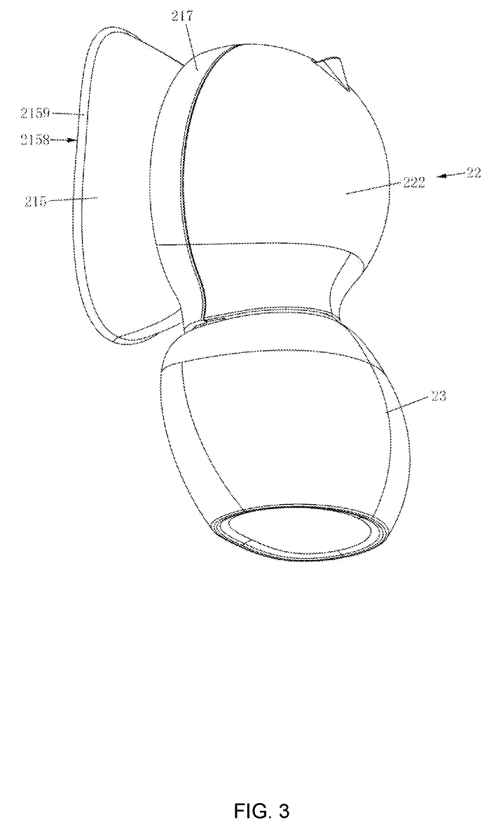

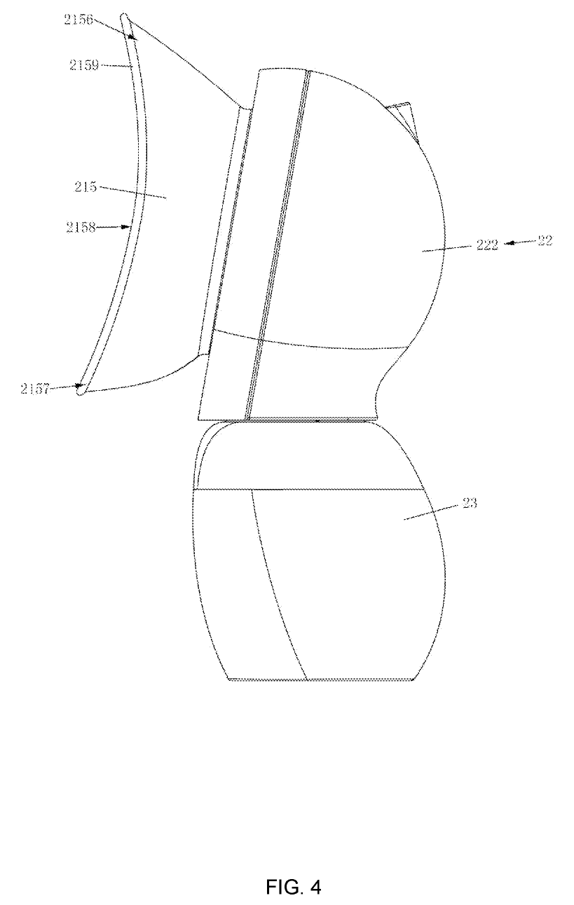

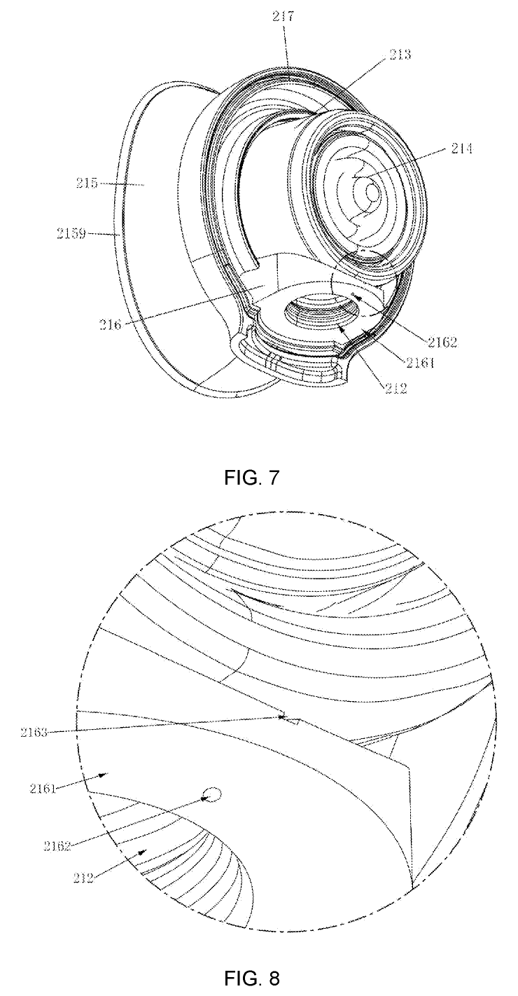

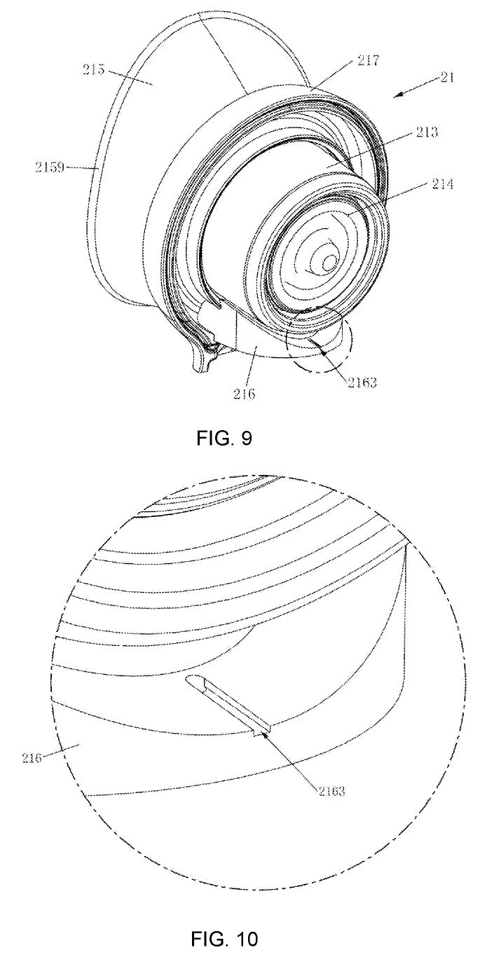

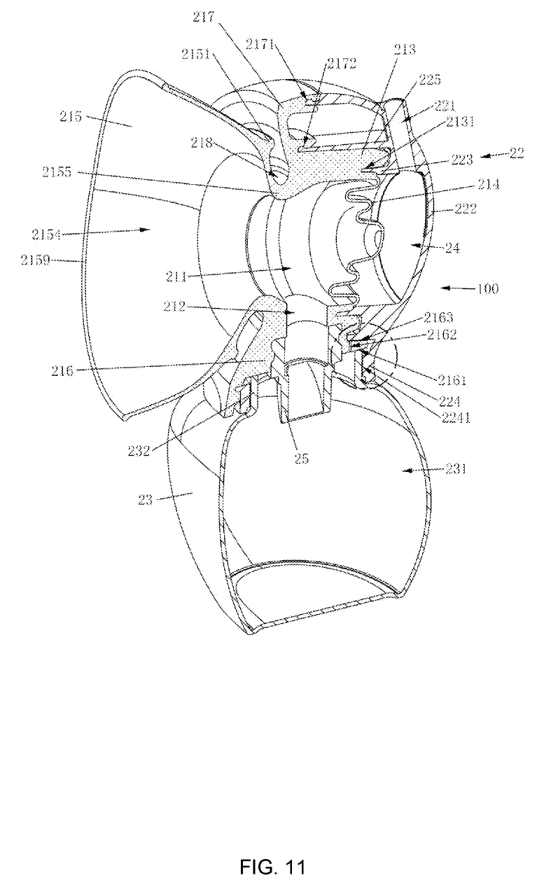

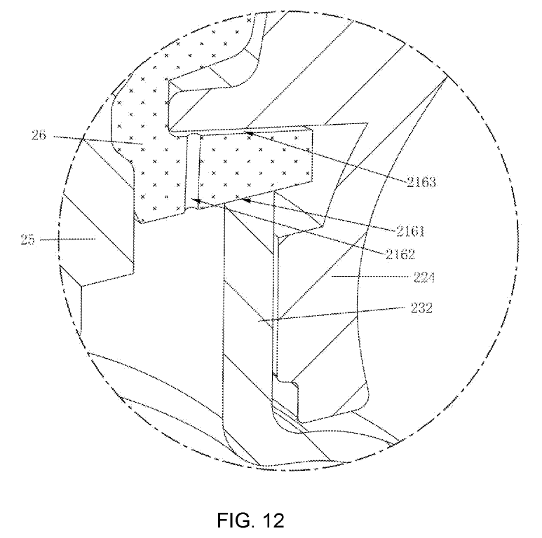

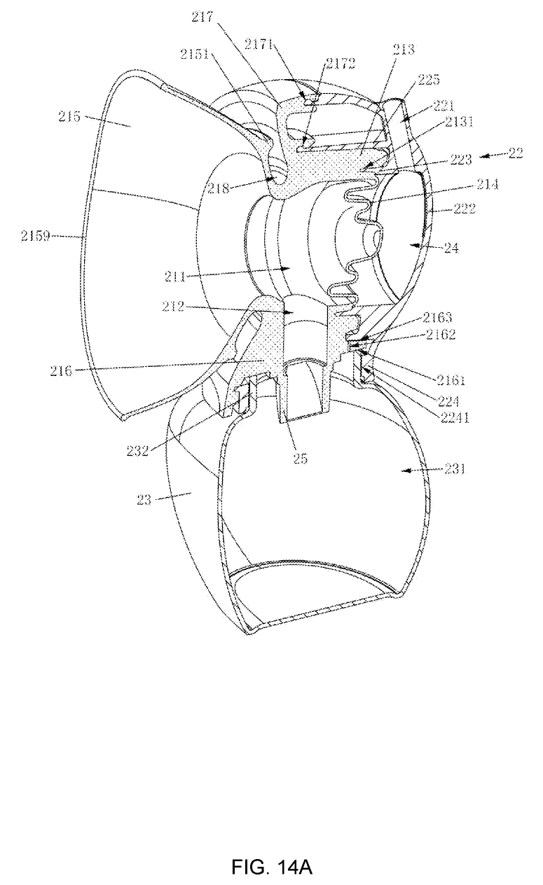

Referring to FIGS. 1 to 13 of the accompanying drawings of this invention, a breast pump according to a preferred embodiment of the present invention will be disclosed and described in the following description, wherein the breast pump comprises a main unit 10 and at least one breast pumping apparatus 20 connected to the main unit 10, the breast pumping apparatus 20 is able to be fitted to the breast, and when the main unit 10 is in operation, the breast pumping apparatus 20 is used to pump out and store milk.

Preferably, the breast pump further comprises an air tube 30, one end of which is connected to the main unit 10 and the other end is connected to the breast pumping apparatus 20, such that the breast pumping apparatus 20 is connected to the main unit 10 through the air tube 30.

More preferably, referring to FIG. 1, the breast pump has two breast pumping apparatuses 20, and the air tube 30 is Y-shaped. The air tube 30 can connect the two pumping devices 20 to the main unit 10 simultaneously, so that the user can use the breast pump of the present invention to pump milk from two breasts at the same time. Optionally, in another specific example of the breast pump of the present invention, both the number of pumping devices 20 and the number of air tubes 30 are two. One air tube 30 is used to connect one breast pumping apparatus 20 to the main unit 10, and the other air tube 30 is used to connect the other breast pumping apparatus 20 to the main unit 10, so that the user can use the breast pump of the present invention to pump milk from both breasts at the same time.

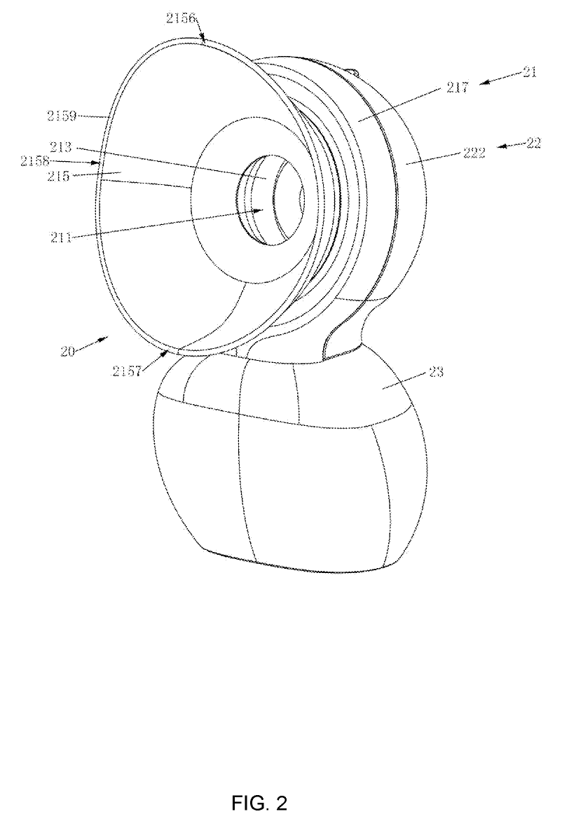

Referring to FIGS. 2 to 13, the breast pumping apparatus 20 comprises a device body 21, a rear cover 22, and a milk bottle 23, and the breast pumping apparatus 20 has a negative pressure chamber 24.

In this specific example of the breast pumping apparatus of the present invention shown in FIGS. 2 to 13, the device body 21 is integrally formed by injection molding. For example, silicone material can be used to form the integral device body 21 by injection molding, or it can be formed by secondary injection molding. The device body 21 has a spatially variable milk suction chamber 211 and a milk discharge channel 212 selectively connected to the milk suction chamber 211. The rear cover 22 has an air channel 221 and is installed at the rear of the device body 21. The negative pressure chamber 24 of the breast pumping apparatus 20 is formed between the device body 21 and the rear cover 22. The air channel 221 of the rear cover 22 is communicated to the negative pressure chamber 24 of the breast pumping apparatus 20, and the pressure state of the milk suction chamber 211 of the device body 21 and the communication state of the milk discharge channel 212 change with the pressure state of the negative pressure chamber 24 of the breast pumping apparatus 20. The milk bottle 23 is detachably mounted to the rear cover 22. When the milk bottle 23 is mounted on the rear cover 22, the rim of the milk bottle 23 abuts against the device body 21. The milk discharge channel 212 connects the milk suction chamber 211 and the milk storage chamber 231 of the milk bottle 23. In the breast pump of the present invention, the device body 21 of the breast pump 20 is integral, which helps to reduce the production cost of the breast pump 20 and improve the production efficiency of the breast pump. Furthermore, since the rim of the milk bottle 23 abuts against the device body 21, on the one hand, the device body 21 can provide pre-pressure to the milk bottle 23 to prevent the milk bottle 23 from loosening; on the other hand, the device body 21 can prevent gaps from forming between the device body 21 and the rim of the milk bottle 23, thereby ensuring that milk is reliably stored in the milk storage chamber 231 of the milk bottle 23.

When a user uses the breast pump, the breast closes the opening of the milk suction chamber 211 of the device body 21, forming a closed space. When the main unit 10 is in operation, it can expel the air in the negative pressure chamber 24 of the breast pump 20 through the air channel 221 of the rear cover 22, thereby increasing the negative pressure in the negative pressure chamber 24. At this time, the negative pressure in the milk suction chamber 211 of the device body 21 increases, and the milk discharge channel 212 prevents the milk suction chamber 211 of the device body 21 from communicating with the milk storage chamber 231 of the milk bottle 23, thus milk is sucked out. The milk is temporarily stored in the milk suction chamber 211 of the device body 21. When the main unit 10 allows air to enter the negative pressure chamber 24 of the breast pump 20 through the air channel 221 of the rear cover 22, the negative pressure in the negative pressure chamber 24 of the breast pump 20 decreases. At this time, the negative pressure in the milk suction chamber 211 of the device body 21 decreases, and the milk discharge channel 212 allows the milk suction chamber 211 of the device body 21 and the milk storage chamber 231 of the milk bottle 23 to communicate. Therefore, milk is allowed to enter the milk storage chamber 231 of the milk bottle 23 through the milk discharge channel 212 of the device body 21. This process is repeated, and the breast pump can help the user express milk.

Further, in this specific example of the breast pump of the present invention shown in FIGS. 1 to 13, the device body 21 comprises a cylindrical body 213, a deformable portion 214, a horn cover 215, and an abutment portion 216. The deformable portion 214 is integrally formed at the rear of the cylindrical body 213, the horn cover 215 is integrally formed at the front of the cylindrical body 213, and the abutment portion 216 is integrally formed at the bottom of the cylindrical body 213. The milk suction chamber 211 of the device body 21 is formed between the cylindrical body 213 and the deformable portion 214, and the milk discharge channel 212 of the device body 21 is extended along the inner wall of the cylindrical body 213 to an abutment surface 2161 of the abutment portion 216.

In other words, in this specific example of the breast pump shown in FIGS. 1 to 13, the cylindrical body 213, the deformable portion 214, the horn cover 215, and the abutment portion 216 of the device body 21 are a single unit and do not need to be assembled with each other.

For example, in some embodiments of the breast pump of the present invention, the silicone material can be injection molded to integrally form the cylindrical body 213, the deformable portion 214, the horn cover 215 and the abutment portion 216 through a single injection molding process.

In some other embodiments of the breast pump of the present invention, the cylindrical body 213 can be injection molded first, and then the deformable portion 214, the horn cover 215, and the abutment portion 216 can be injection molded. The deformable portion 214 is integrally formed at the rear of the cylindrical body 213, the horn cover 215 is integrally formed at the front of the cylindrical body 213, and the abutment portion 216 is integrally formed at the bottom of the cylindrical body 213.

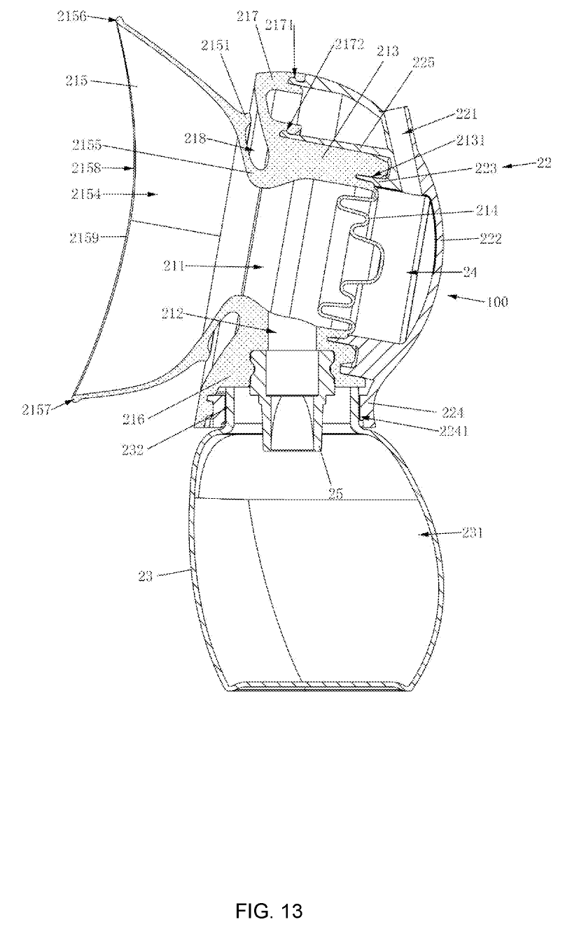

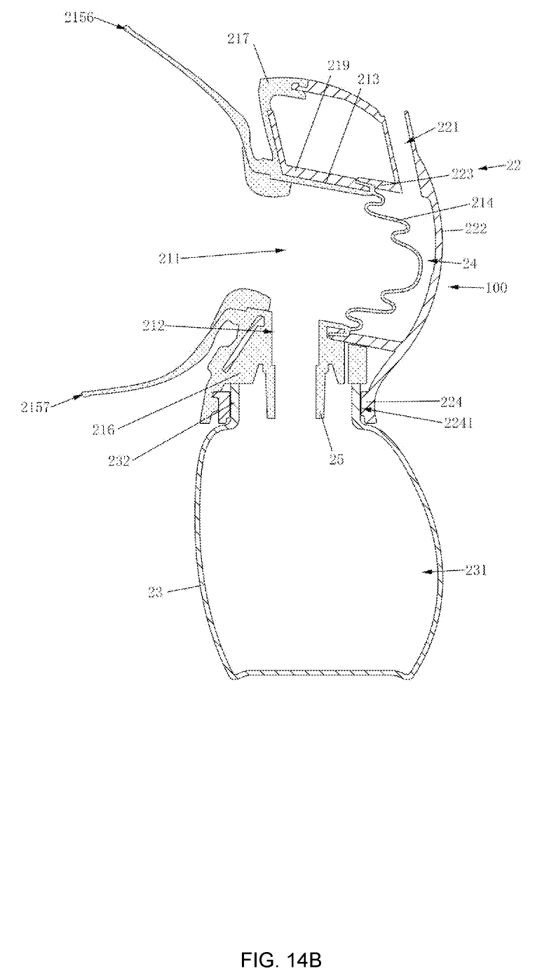

Preferably, referring to FIGS. 11 and 13, the inner wall of the device body 21, which forms the milk suction chamber 211, is inclinedly extended from the connection position of the horn cover 215 and the cylindrical body 213 toward the deformable portion 214. When the user covers the breast with the horn cover 215, the connection position of the horn cover 215 and the cylindrical body 213 abuts against the areola. Since the inner wall of the device body 21, which forms the milk suction chamber 211, is inclined, on the one hand, the inner wall of the device body 21, which forms the milk suction chamber 211, can guide the sucked milk to the milk discharge channel 212 of the device body 21, so that the milk can be discharged smoothly. On the other hand, the connection position of the horn cover 215 and the cylindrical body 213 can prevent the milk from flowing back, so that the user can use the milk suction device 20 of the present invention to suckle.

The rear cover 22 is installed on the cylindrical body 213, and the negative pressure chamber 24 of the breast pump 20 is formed between the deformable portion 214 and the rear cover 22. When the user uses the breast pump, the breast closes the opening of the milk suction chamber 211 of the device body 21, making the milk suction chamber 211 of the device body 21 a closed space. At this time, the horn cover 215 causes the device body 21 to attach to the breast. As the air in the negative pressure chamber 24 of the breast pump 20 is discharged through the air channel 221 of the rear cover 22, the deformable portion 214 deforms toward the rear cover 22, causing the space of the milk suction chamber 211 of the device body 21 to increase. Correspondingly, the negative pressure in the milk suction chamber 211 of the device body 21 increases, and the milk discharge channel 212 of the device body 21 prevents the milk suction chamber 211 of the device body 21 from communicating with the milk storage chamber 231 of the milk bottle 23. Milk is drawn out and temporarily stored in the milk suction chamber 211 of the device body 21. As external air enters the negative pressure chamber 24 of the milk suction device 20 through the air channel 221 of the rear cover 22, the deformable portion 214 deforms away from the rear cover 22, resulting in a reduction in the space of the milk suction chamber 211 of the device body 21. Correspondingly, the negative pressure in the milk suction chamber 211 of the device body 21 decreases, and the milk discharge channel 212 of the device body 21 allows the milk suction chamber 211 of the device body 21 and the milk storage chamber 231 of the milk bottle 23 to communicate. The milk temporarily stored in the milk suction chamber 211 of the device body 21 enters the milk storage chamber 231 of the milk bottle 23 through the milk discharge channel 212 of the device body 21.

When the milk bottle 23 is installed on the rear cover 22, the rim of the milk bottle 23 applies pressure to the abutment portion 216 after it is in contact with the abutment surface 2161 of the abutment portion 216, causing the abutment portion 216 to deform slightly. In this way, on the one hand, the device body 21 can provide pre-pressure to the milk bottle 23 to prevent the milk bottle 23 from loosening, and on the other hand, the device body 21 can prevent gaps from forming between the abutment surface 2161 of the abutment portion 216 and the rim of the milk bottle 23, so that the milk is reliably stored in the milk storage chamber 231 of the milk bottle 23.

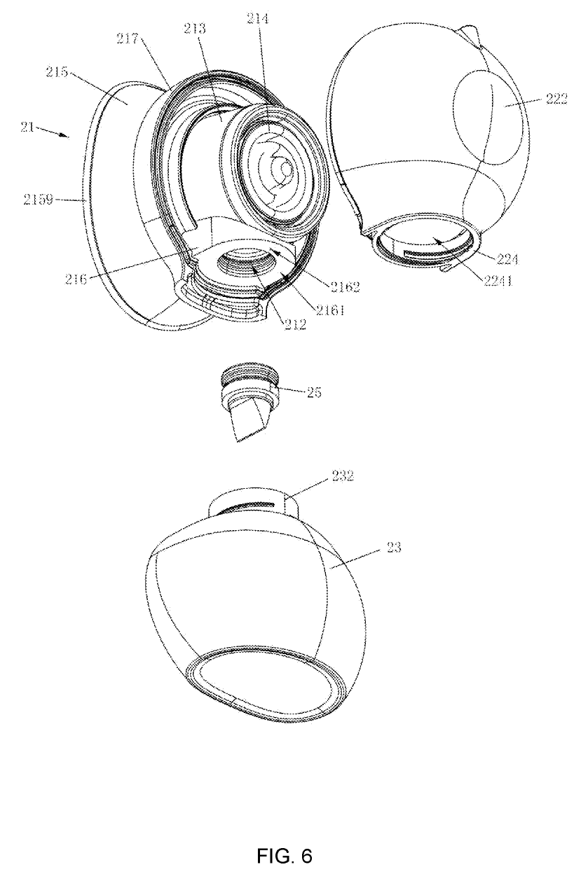

Referring to FIGS. 6 to 12, the abutment portion 216 of the device body 21 has a vent hole 2162 which is extended from the abutment surface 2161 to the side of the abutment portion 216 opposite to the abutment surface 2161. That is, the vent hole 2162 of the abutment portion 216 penetrates the upper and lower sides of the abutment portion 216. After the milk bottle 23 is installed on the rear cover 22 and the rim of the milk bottle 23 abuts against the abutment surface 2161 of the abutment portion 216, the vent hole 2162 of the abutment portion 216 is located inside the rim of the milk bottle 23. In this way, the vent hole 2162 of the abutment portion 216 can connect the milk storage chamber 231 of the milk bottle 23 with the external environment. Thus, during the milk feeding process, the air in the milk storage chamber 231 of the milk bottle 23 can be discharged through the vent hole 2162 of the abutment portion 216, so that the milk temporarily stored in the milk suction chamber 211 of the device body 21 can be smoothly discharged into the milk storage chamber 231 of the milk bottle 23 through the milk discharge channel 212 of the device body 21.

Preferably, the abutment portion 216 of the device body 21 has an exhaust groove 2163. The exhaust groove 2163 is provided on the side of the abutment portion 216 away from the abutment surface 2161, and the exhaust groove 2163 is extended from the vent hole 2162 to the edge of the abutment portion 216. In this way, after the rear cover 22 is installed on the device body 21, the exhaust groove 2163 of the abutment portion 216 allows an exhaust channel to be formed between the device body 21 and the rear cover 22. The exhaust channel connects the vent hole 2162 of the abutment portion 216 and the external environment, so that during the milk feeding process, the air in the milk storage chamber 231 of the milk bottle 23 can be discharged through the vent hole 2162 of the abutment portion 216 and the exhaust channel.

Optionally, in other examples of the breast pump of the present invention, the exhaust groove 2163 may also be provided on the rear cover 22. After the rear cover 22 is installed on the device body 21, the vent hole 2162 of the abutment portion 216 and the exhaust groove 2163 of the rear cover 22 are communicated and connected. In this way, the exhaust groove 2163 of the rear cover 22 allows the venting channel to be formed between the device body 21 and the rear cover 22, and the venting channel connects the vent hole 2162 of the abutment portion 216 and the external environment. Thus, during the breast pumping process, the air in the milk storage chamber 231 of the milk bottle 23 can be discharged through the vent hole 2162 of the abutment portion 216 and the venting channel.

Optionally, in other examples of the breast pump of the present invention, the abutment portion 216 of the device body 21 may also lack the vent hole 2162, and the exhaust groove 2163 of the abutment portion 216 is provided on the abutment surface 2161 of the abutment portion 216, and the exhaust groove 2163 is extended from the edge of the abutment portion 216 toward the milk discharge channel 212. Thus, when the milk bottle 23 is mounted on the rear cover 22 and the rim of the milk bottle 23 abuts against the abutment portion 216, the exhaust groove 2163 is extended from the edge of the abutment portion 216 toward the milk discharge channel 212. After the contact surface 2161 is engaged, the exhaust groove 2163 of the abutment portion 216 allows the milk storage chamber 231 of the milk bottle 23 to communicate with the external environment. Thus, during the milk feeding process, the air in the milk storage chamber 231 of the milk bottle 23 can be discharged through the exhaust groove 2163 of the abutment portion 216, so that the milk temporarily stored in the milk suction chamber 211 of the device body 21 can be smoothly discharged into the milk storage chamber 231 of the milk bottle 23 through the milk discharge channel 212 of the device body 21.

Referring to FIGS. 2 to 7, 9, 11 and 13, the device body 21 comprises a panel portion 217 which is integrally formed on the cylindrical body 213 and surrounds the middle portion of the cylindrical body 213. It is understood that in some embodiments of the breast pump of the present invention, the panel portion 217 can be integrally formed with the cylindrical body 213, the deformable portion 214, the horn cover 215, and the abutment portion 216 through an integral injection molding process. The edge of the rear cover 22 is mounted to the edge of the panel portion 217, thereby reliably mounting the rear cover 22 to the device body 21.

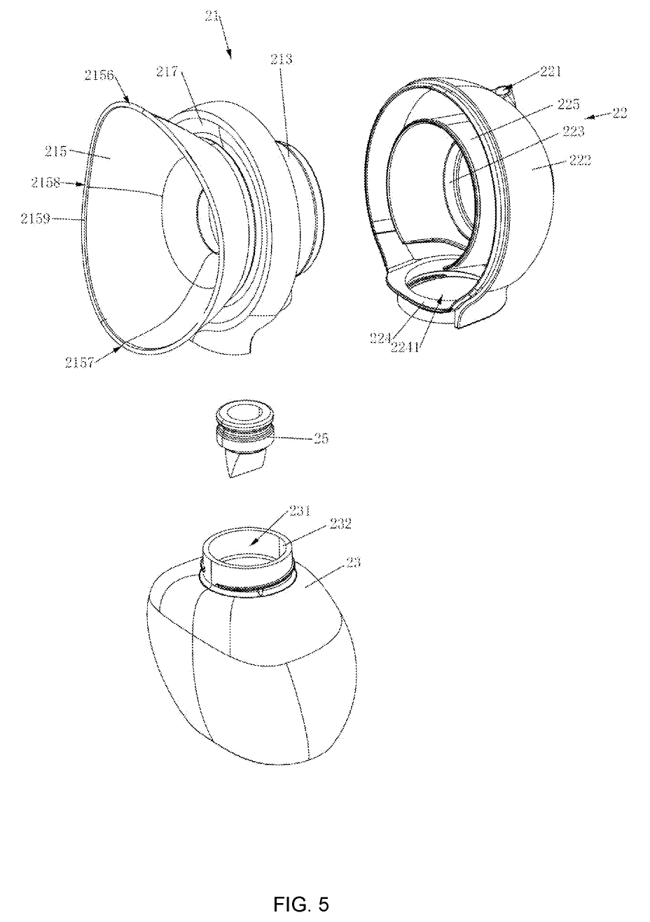

Referring to FIGS. 5, 6, 11, and 13, the rear cover 22 comprises a cover shell 222, an inner abutment ring arm 223, and an assembly ring 224. The inner abutment ring arm 223 is extended integrally into the middle of the cover shell 222, and the assembly ring 224 is integrally formed at the bottom of the cover shell 222. When the rear cover 22 is installed on the device body 21, the edge of the cover shell 222 is installed on the edge of the panel portion 217. The inner abutment ring arm 223 sealably abuts against the rear portion of the cylindrical body 213 to form the negative pressure chamber 24 of the breast pumping apparatus 20 between the rear cover 22 and the deformable portion 214. The assembly ring 224 is located below the abutment portion 216, and the milk discharge channel 212 of the device body 21 corresponds to a ring channel 2241 of the assembly ring 224.

Referring to FIGS. 11 and 13, the panel portion 217 has an outer insertion groove 2171, and the tail end of the cylindrical body 213 has a cylinder insertion groove 2131. When the rear cover 22 is installed on the device body 21, the edge of the cover shell 222 is inserted into the outer insertion groove 2171 of the panel portion 217, and the inner abutment ring arm 223 is inserted into the cylinder insertion groove 2131 of the cylindrical body 213. In this way, not only can the rear cover 22 be reliably installed on the device body 21, but the sealing of the negative pressure cavity 24 formed between the rear cover 22 and the deformable portion 214 can also be guaranteed.

The mouth 232 of the milk bottle 23 passes through the ring channel 2241 of the assembly ring 224 and is installed to the assembly ring 224, and the rim of the milk bottle 23 abuts against the abutment surface 2161 of the abutment portion 216. In this specific example of the breast pump of the present invention shown in FIGS. 1 to 13, the mouth 232 of the milk bottle 23 is screwed onto the assembly ring 224. When the mouth 232 of the milk bottle 23 is screwed onto the assembly ring 224, pressure can be applied to the abutment surface 2161 of the abutment portion 216, causing slight deformation of the abutment surface 2161. This allows the device body 21 to provide pre-pressure to the milk bottle 23, preventing the milk bottle 23 from loosening. At the same time, the device body 21 can prevent gaps from forming between the abutment surface 2161 of the abutment portion 216 and the mouth rim of the milk bottle 23, ensuring that milk is reliably stored in the milk storage chamber 231 of the milk bottle 23.

Furthermore, the rear cover 22 comprises an outer abutment ring arm 225 which is integrally extended from the middle of the cover shell 222 and is located outside the inner abutment ring arm 223. The outer abutment ring arm 225 is mounted on the inner side of the panel portion 217, thus allowing the rear cover 22 to be reliably mounted to the device body 21. Preferably, the panel portion 217 has an inner insertion groove 2172, and when the rear cover 22 is mounted to the device body 21, the outer abutment ring arm 225 is inserted into the inner insertion groove 2172 of the panel portion 217.

Referring to FIGS. 5, 6, 11 and 13, the breast pumping apparatus 20 comprises a duckbill valve 25 which is installed on the abutment portion 216 of the device body 21 to control the connection state of the milk discharge channel 212 of the device body 21. Specifically, when a user uses the breast pump to express milk, when the negative pressure in the milk suction chamber 211 of the device body 21 increases as the negative pressure in the negative pressure chamber 24 of the breast pump 20 increases, the duckbill valve 25 prevents the milk suction chamber 211 of the device body 21 from communicating with the milk storage chamber 231 of the milk bottle 23. When the negative pressure in the milk suction chamber 211 of the device body 21 decreases as the negative pressure in the negative pressure chamber 24 of the breast pump 20 decreases, the duckbill valve 25 allows the milk suction chamber 211 of the device body 21 to communicate with the milk storage chamber 231 of the milk bottle 23.

Referring again to FIGS. 11 and 13, a gap 218 exists between the horn cover 215 and the panel portion 217 of the device body 21, allowing the deformed horn cover 215 to flip towards the panel portion 217. The horn cover 215 has a support ring 2151 facing the panel portion 217, allowing the support ring 2151 to abut against the panel portion 217. In this way, during milk expression, the support ring 2151 supports the lower side of the horn cover 215 and the panel portion 217, preventing the horn cover 215 from sagging and causing the nipple to lose balance within the milk suction chamber 211 of the device body 21, thereby preventing milk leakage. Meanwhile, since the support ring 2151 is annular, the support ring 2151 itself can maintain the shape of the horn cover 215, reduce the deformation range of the horn cover 215, so that no gap will be generated between the breast and the horn cover 215 when the user uses the breast pump.

Referring again to FIGS. 1 to 13, according to another aspect of the present invention, the breast pumping apparatus 20 of the present invention comprises a milk suction unit 100 and a horn cover 215 disposed on the milk suction unit 100.

The milk suction unit 100 has a milk suction chamber 211, a milk discharge channel 212 selectively connected to the milk suction chamber 211, and a negative pressure chamber 24 independent of the milk suction chamber 211. The pressure state of the milk suction chamber 211 and the connection state of the milk discharge channel 212 change with the pressure state of the negative pressure chamber 24. When the negative pressure in the negative pressure chamber 24 increases, the negative pressure in the milk suction chamber 211 increases, and the milk discharge channel 212 closes. When the negative pressure in the negative pressure chamber 24 decreases, the negative pressure in the milk suction chamber 211 decreases, and the milk discharge channel 212 opens.

The horn cover 215 has a cover space 2154 and an abutting portion 2155. The cover space 2154 of the horn cover 215 is communicated to the milk suction chamber 211 of the milk suction unit 100. The abutting portion 2155 is located at the connection position between the cover space 2154 and the milk suction chamber 211. The surface of the horn cover 215 that defines the cover space 2154 is a concave arc surface, and the part of the horn cover 215 that defines the cover space 2154 can be flipped backward. In this way, the user can first flip the part of the horn cover 215 that defines the cover space 2154 backward, then align the nipple with the milk suction chamber 211, and then restore the horn cover 215. Thus, the horn cover 215 can self-adhere to the breast. Because the surface of the horn cover 215 that defines the cover space 2154 is a concave arc surface, on the one hand, the shape of the surface of the horn cover 215 that defines the cover space 2154 matches the shape of the breast; on the other hand, the opening size of the cover space 2154 of the horn cover 215 can be controlled to a reasonable size. When the breast is received in the cover space 2154 of the horn cover 215, the areola abuts against the abutting portion 2155 of the horn cover 215, the surface of the horn cover 215 that defines the cover space 2154 fits tightly against the breast, and the opening edge of the horn cover 215 catches the base of the breast, so that the horn cover 215 can self-adhere to the breast, achieving "naked pumping". In other words, during the pumping process, the user does not need to hold the breast pumping apparatus 20 by hand, nor does the user need underwear for support, thereby improving the convenience and comfort of the user when using the breast pump. Preferably, the horn cover 215 is flexible. For example, the horn cover 215 may be made of silicone material so that the horn cover 215 is flexible.

Specifically, the milk suction unit 100 comprises the device body 21 and the rear cover 22. The device body 21 further comprises the cylindrical body 213 and the deformable portion 214. The deformable portion 214 is integrally formed at the rear of the cylindrical body 213. The milk suction chamber 211 is formed between the cylindrical body 213 and the deformable portion 214. The milk discharge channel 212 is formed in the cylindrical body 213. The rear cover 22 is installed in the cylindrical body 213. The negative pressure chamber 24 is formed between the deformable portion 214 and the rear cover 22. When the negative pressure in the negative pressure chamber 24 increases, the deformable portion 214 deforms toward the rear cover 22. At this time, the space in the milk suction chamber 211 increases and the negative pressure increases. Correspondingly, when the negative pressure in the negative pressure chamber 24 decreases, the deformable portion 214 deforms away from the rear cover 22. At this time, the space in the milk suction chamber 211 decreases and the negative pressure decreases.

In one specific example of the breast pump of the present invention, the horn cover 215 may be integrally formed on the front of the cylindrical body 213 to be disposed on the milk suction unit 100. Optionally, in other specific examples of the breast pump of the present invention, the horn cover 215 may be mounted on the front of the cylindrical body 213 to be disposed on the milk suction unit 100.

The top of the horn cover 215 has a top protrusion 2156, the bottom of the horn cover 215 has a bottom protrusion 2157, and the opposite sides of the horn cover 215 each have a contraction portion 2158. The vertical distance between the top protrusion 2156 and the abutting portion 2155 is greater than the vertical distance between the bottom protrusion 2157 and the abutting portion 2155, and the vertical distance between the bottom protrusion 2157 and the abutting portion 2155 is greater than the vertical distance between the contraction portion 2158 and the abutting portion 2155. In this way, on the one hand, the shape of the horn cover 215 can match the shape of the breast, and on the other hand, after the user attaches the horn cover 215 to the breast, the top protrusion 2156 and the bottom protrusion 2157 extend toward the root of the breast from the upper and lower parts of the breast respectively, so as to increase the contact surface between the horn cover 215 and the breast, thereby allowing the horn cover 215 to be firmly attached to the breast.

Optionally, in other examples of the breast pump of the present invention, the top protrusion 2156 is formed on the top of the horn cover 215, and the bottom protrusion 2157 is not formed on the bottom of the horn cover 215. In this case, the contraction portions 2158 are not formed on the opposite sides of the horn cover 215. It is understood that since the top protrusion 2156 is formed on the top of the horn cover 215, after the user attaches the horn cover 215 to the breast, the top protrusion 2156 extends from the upper part of the breast to the root of the breast to increase the contact surface between the horn cover 215 and the breast, thereby allowing the horn cover 215 to be firmly attached to the breast.

Preferably, the curvature of the bottom of the horn cover 215 is greater than that of the top, so that the shape of the horn cover 215 can better match the shape of the breast.

In addition, the device body 21 may also comprise the abutment portion 216 which is integrally formed at the bottom of the cylindrical body 213, and the milk discharge channel 212 is extended from the inner wall of the cylindrical body 213 to the abutment surface 2161 of the abutment portion 216. The milk suction unit 100 comprises the milk bottle 23 which is detachably mounted on the rear cover 22. After the milk bottle 23 is mounted on the rear cover 22, the rim of the milk bottle 23 abuts against the abutment surface 2161 of the abutment portion 216, causing the abutment portion 216 to deform slightly. In this way, on the one hand, the device body 21 can provide pre-pressure to the milk bottle 23 to prevent the milk bottle 23 from loosening; on the other hand, the device body 21 can prevent gaps from forming between the abutment surface 2161 of the abutment portion 216 and the rim of the milk bottle 23, thereby ensuring that milk is reliably stored in the milk storage chamber 231 of the milk bottle 23.

In addition, the milk suction unit 100 also comprises the duckbill valve 25 which is disposed on the abutment portion 216 of the device body 21 to control the connection state of the milk discharge channel 212 of the milk suction unit 100.

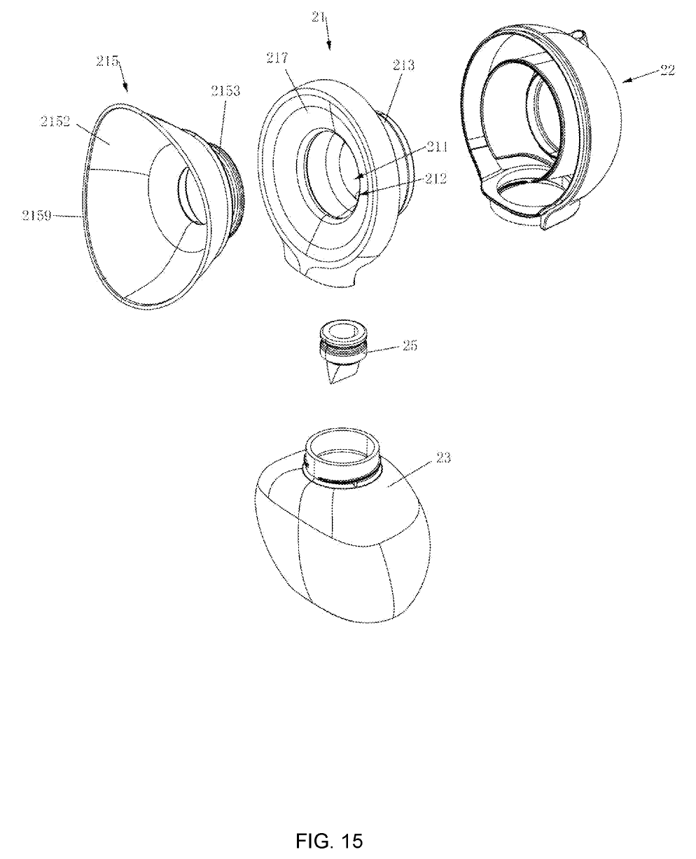

FIG. 14A illustrates a modified example of the breast pumping apparatus 20 of the breast pump according to the above-described preferred embodiment of the present invention. Unlike the breast pumps shown in FIGS. 1 to 13, in this specific example of the breast pumping apparatus 20 of the breast pump shown in FIG. 14A, the duckbill valve 25 and the device body 21 are integral. The silicone material can be injection molded into an integral cylindrical body 213, the deformable portion 214, the horn cover 215, the abutment portion 216, and the duckbill valve 25 through a single injection molding process.

FIG. 14B illustrates a modified example of the breast pumping apparatus 20 of the breast pump according to the above-described preferred embodiment of the present invention. Unlike the breast pumps shown in FIGS. 1 to 13, in this specific example of the breast pumping apparatus 20 shown in FIG. 14B, the device body 21 further comprises an annular frame 219 which is disposed on the cylindrical body 213 to maintain the shape of the cylindrical body 213 and ensure that the cylindrical body 213 will not deform when it has a thinner size. This helps to reduce the overall weight of the breast pumping apparatus 20, so that the horn cover 215 can be more firmly attached to the breast.

It is worth mentioning that the specific manner in which the annular frame 219 is set in the cylindrical body 213 is not limited in the breast pumping apparatus 20 of the present invention. For example, in the specific example of the breast pumping apparatus 20 of the present invention shown in FIG. 14B, the encapsulation process can be used to set the annular frame 219 in the cylindrical body 213, and the encapsulation process can make the annular frame 219 completely embedded inside the cylindrical body 213, making the annular frame 219 visually invisible. Optionally, the encapsulation process can also make the annular frame 219 partially embedded in the cylindrical body 213.

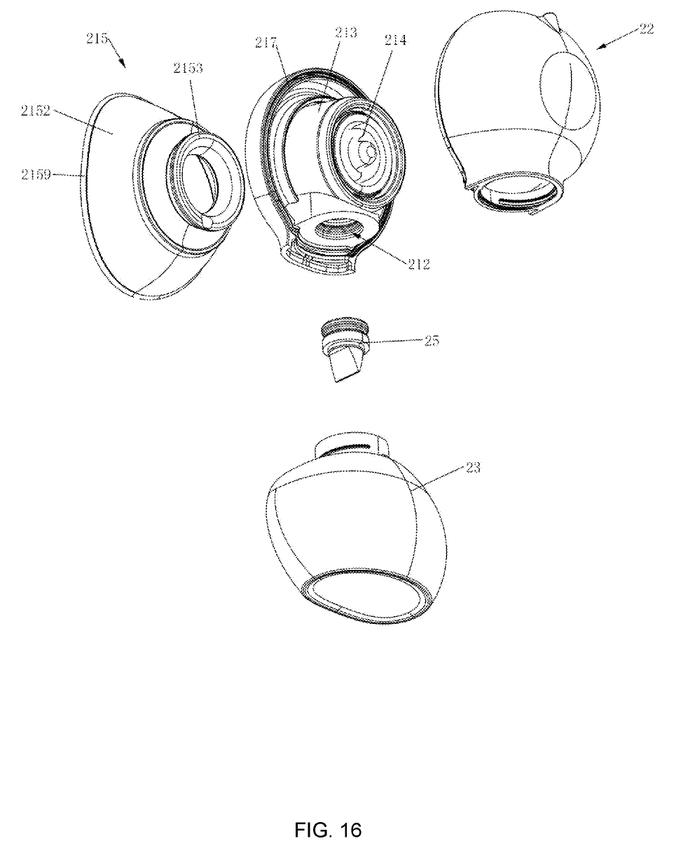

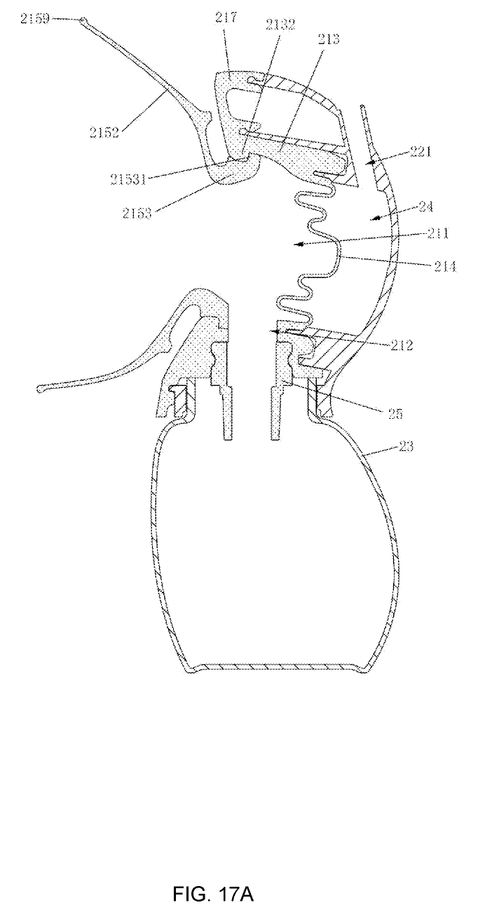

FIGS. 15 to 17A show another alternative mode of the breast pumping apparatus 20 of the preferred embodiment of the present invention. Unlike the breast pumping apparatus 20 shown in FIGS. 1 to 13, in this specific example of the breast pumping apparatus 20 of the breast pump shown in FIGS. 15 to 17A, the cylindrical body 213, the deformable portion 214, and the abutment portion 216 of the device body 21 are integrally formed. The deformable portion 214 is integrally formed at the rear of the cylindrical body 213, the abutment portion 216 is integrally formed at the bottom of the cylinder 21, the milk suction chamber 211 is formed between the cylindrical body 213 and the deformable portion 214, the milk discharge channel 212 extends from the inner wall of the cylindrical body 213 to the abutment surface 2161 of the abutment portion 216, and the horn cover 215 is installed at the front of the cylindrical body 213. In other words, in this specific example of the breast pumping apparatus 20 of the present invention shown in FIGS. 15 to 17A, the horn cover 215 can be removed from the front of the cylindrical body 213, so that, on the one hand, the horn cover 215 can be washed separately, and on the other hand, the horn cover 215 can be replaced.

Specifically, the horn cover 215 comprises a horn portion 2152 and a mounting portion 2153 extended rearward from the rear of the horn portion 2152. The mounting portion 2153 is mounted on the front of the cylindrical body 213. The horn portion 2152 can cover the user's breast. The cover space 2154 of the horn cover 215 is formed in the horn portion 2152, and the abutting portion 2155 is formed at the connection position between the horn portion 2152 and the mounting portion 2153. Preferably, the mounting portion 2153 is extended rearward and outward from the rear of the horn portion 2152, such that the inner wall of the mounting portion 2153 is inclined, extending obliquely from the connection position of the horn portion 2152 and the mounting portion 2153 toward the milk suction chamber 211 of the device body 21. When the user covers the breast with the horn portion 2152 of the horn cover 215, the connection position of the horn portion 2152 and the mounting portion 2153 of the horn cover 215 abuts against the areola. Since the inner wall of the mounting portion 2153 of the horn cover 215 is inclined, on the one hand, the mounting portion 2153 of the horn cover 215 can guide the sucked milk to the milk discharge channel 212 of the device body 21, so that the milk can be discharged smoothly. On the other hand, the connection position between the horn portion 2152 and the mounting portion 2153 of the horn cover 215 can prevent the milk from flowing back, so that the user can use the milk suction device 20 of the present invention to suck.

More specifically, the cylindrical body 213 has a cylindrical body hook 2132, and the mounting portion 2153 of the horn cover 215 is provided with a mounting hook 21531, the mounting hook 21531 protruding circumferentially from the mounting portion 2153, wherein the mounting hook 21531 of the mounting portion 2153 hooks the cylindrical body hook 2132 of the cylindrical body 213 to reliably install the mounting portion 2153 at the front of the cylindrical body 213, thereby reliably installing the horn cover 215 onto the cylindrical body 213.

It is understood that the top protrusion 2156, the bottom protrusion 2157, and each of the contraction portions 2158 of the horn cover 215 are respectively formed on the horn portion 2152.

Referring again to FIGS. 15 to 17A, the horn cover 215 comprises a tapered portion 2159 which is disposed on the outer edge of the horn portion 2152. The thickness of the tapered portion 2159 is greater than the thickness of the periphery of the opening of the horn portion 2152. In this way, on the one hand, the tapered portion 2159 can prevent the periphery of the opening of the horn portion 2152 from being too thin and breaking. On the other hand, the tapered portion 2159 can increase the strength of the periphery of the opening of the horn portion 2152, which is beneficial to maintaining the periphery shape of the opening of the horn portion 2152, thereby allowing the horn cover 215 to reliably self-adhere to the breast.

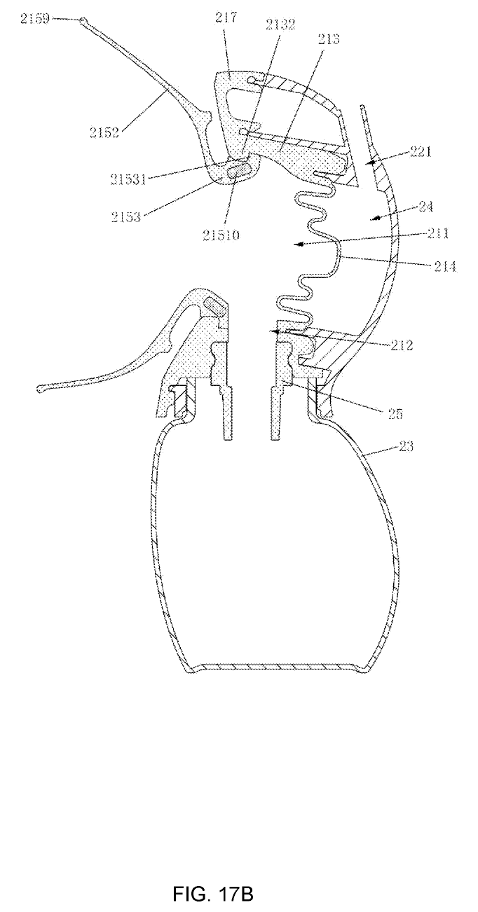

FIG. 17B shows a modified example of the breast pumping apparatus 20 of the present invention. Unlike the breast pumping apparatus 20 shown in FIGS. 15 to 17A, in this specific example of the breast pumping apparatus 20 shown in FIG. 17B, the horn cover 215 comprises a ring frame 21510 which is disposed on the mounting portion 2153 to maintain the shape of the mounting portion 2153, so that the mounting portion 2153 can be reliably installed on the cylindrical body 213.

It is worth mentioning that the specific manner in which the ring frame 21510 is set in the assembly portion 2153 is not limited in the breast pumping apparatus 20 of the present invention. For example, the insert injection molding process can embed the ring frame 21510 inside the assembly portion 2153 to set the ring frame 21510 in the assembly portion 2153. Another example is the overmolding process, which can set the ring frame 21510 in the assembly portion 2153.

In one example of the breast pumping apparatus 20 of the present invention, the hardness of the horn portion 2152 of the horn cover 215 is less than the hardness of the mounting portion 2153, so that the horn portion 2152 is easily deformable to facilitate the user's use of the breast pumping apparatus 20, and the mounting portion 2153 is not easily deformable, so that the mounting portion 2143 can be reliably installed on the cylindrical body 213. In order to make the hardness of the mounting portion 2153 of the horn cover 215 greater than that of the horn portion 2152, in this specific example of the breast pumping apparatus 20 of the present invention shown in FIG. 17B, both the mounting portion 2153 and the horn portion 2152 are made of silicone material, and the ring frame 21510 is provided on the mounting portion 2153. Thus, the hardness of the mounting portion 2153 is greater than that of the horn portion 2152. In other examples of the breast pumping apparatus 20 of the present invention, the mounting portion 2153 may be made of plastic material and the horn portion 2152 may be made of silicone material. Thus, the hardness of the mounting portion 2153 is greater than that of the horn portion 2152.

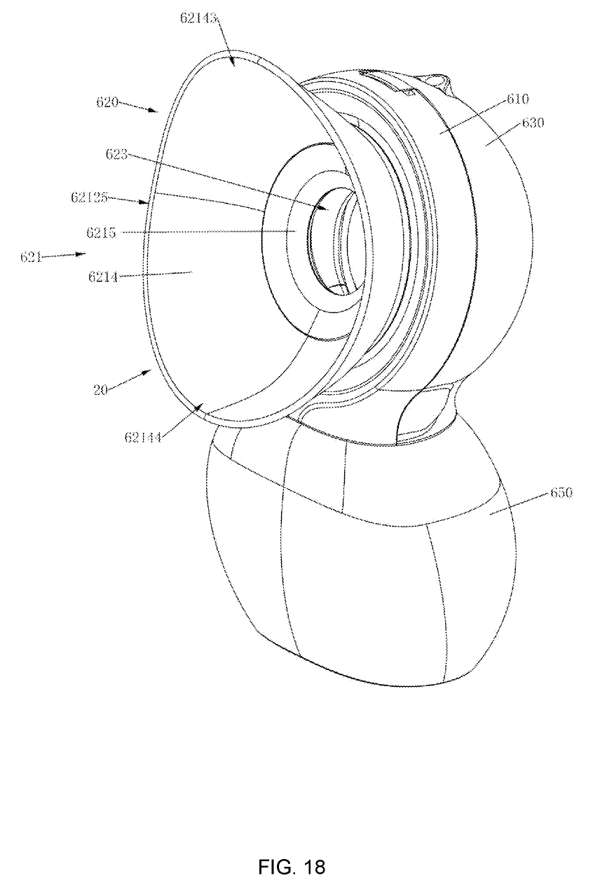

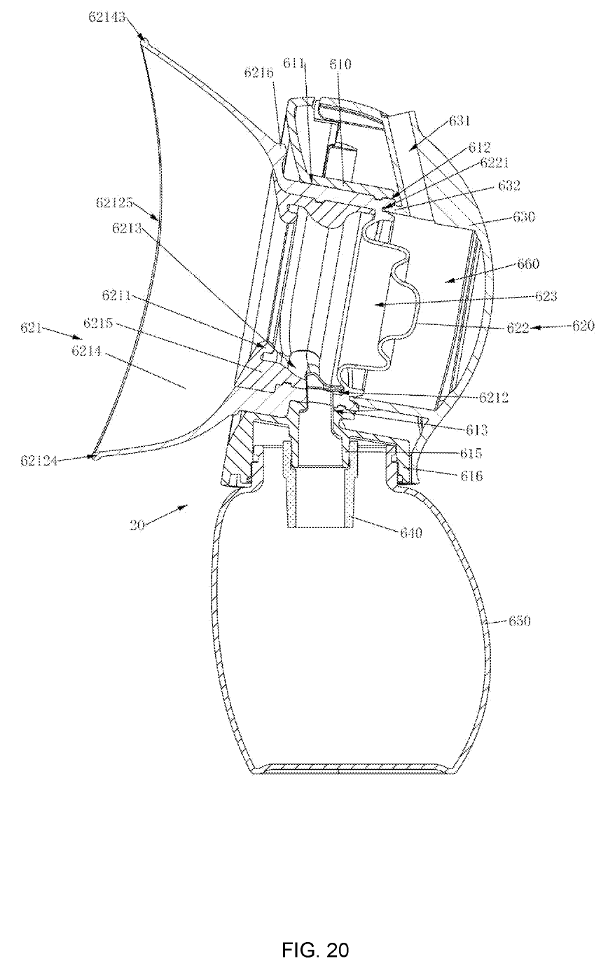

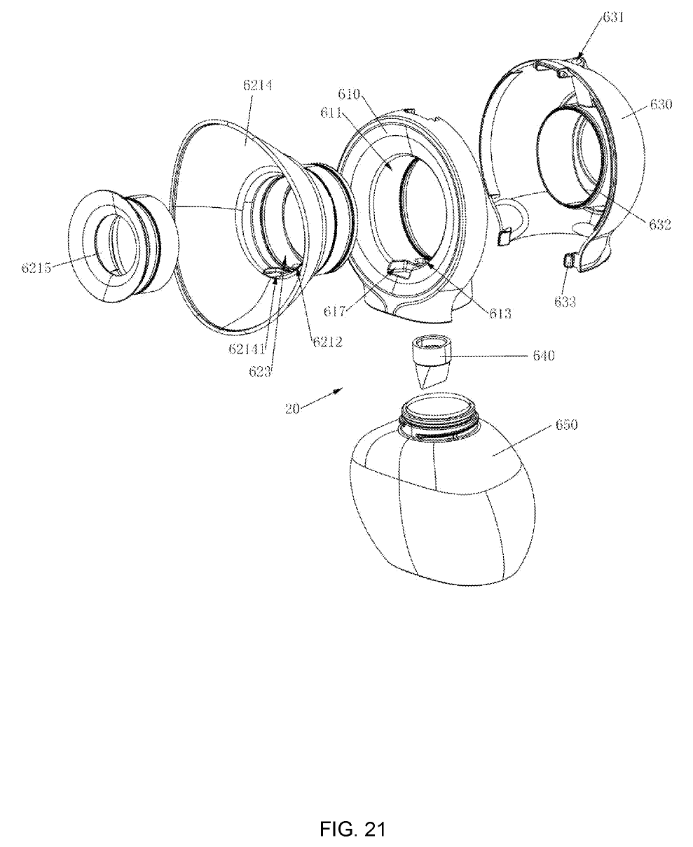

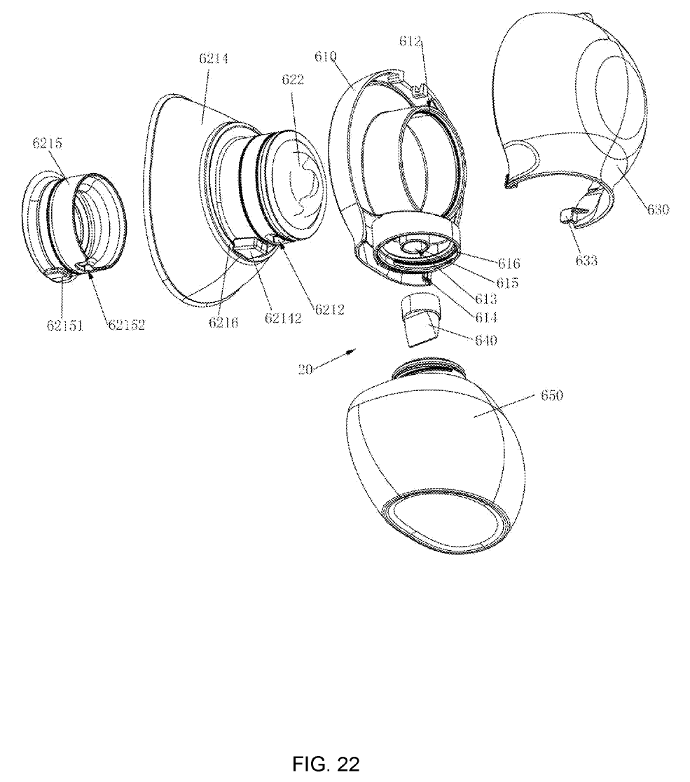

FIGS. 18 to 22 show a breast pumping apparatus 20 according to another preferred embodiment, wherein the breast pumping apparatus 20 comprises a three-way component 610, a device body 620, a rear cover 630, a duckbill valve 640 and a milk bottle 650, and the breast pumping apparatus 20 has a negative pressure chamber 660.

The three-way component 610 has a front opening 611, a rear opening 612, and a bottom opening 613. In this specific example of the breast pumping apparatus 20 of the present invention shown in FIGS. 18 to 22, the three-way component 610 can be an injection molded part, and the front opening 611, the rear opening 612, and the bottom opening 613 of the three-way component 610 are formed simultaneously during injection molding.

The device body 620 comprises a cover portion 621, a deformable portion 622, and a milk suction chamber 623. The cover portion 621 has an areola abutment portion 6211, a milk discharge channel 6212, and a guide groove 6213. The deformable portion 622 is disposed on the cover portion 621; for example, the deformable portion 622 can be disposed at the tail end of the cover portion 621. The main body 620 forms the milk suction chamber 623 between the cover portion 621 and the deformable portion 622. The size of the milk suction chamber 623 varies with the size of the deformable portion 622. The areola abutment portion 6211 is located in the middle section of the cover portion 621. The milk discharge channel 6212 is communicated to the milk suction chamber 623 at the rear of the cover portion 621. The guide groove 6213 is inclinedly extended from the areola abutment portion 6211 to the milk discharge channel 6212, that is, the height of the end of the guide groove 6213 adjacent to the areola abutment portion 6211 is higher than the height of the end of the guide groove 6213 adjacent to the milk discharge channel 6212. The rear of the cover portion 621 is installed on the three-way component 610 through the front opening 611, and the deformable portion 622 is extended out of the three-way component 610 through the rear opening 612. The milk discharge channel 6212 of the cover portion 621 is communicated to the bottom opening 613 of the three-way component 610.

The rear cover 630 has an air channel 631. The rear cover 630 is installed on the three-way component 610. The periphery of the deformable portion 622 is clamped between the three-way component 610 and the rear cover 630. The negative pressure chamber 660 of the breast pumping apparatus 20 is formed between the deformable portion 622 and the rear cover 630. The air channel 631 of the rear cover 630 is communicated to the negative pressure chamber 660 of the breast pumping apparatus 20. Understandably, when the air in the negative pressure chamber 660 of the breast pump 20 is discharged through the air channel 631 of the rear cover 630, the deformable portion 622 can deform toward the rear cover 630, at which time the spatial size of the milk suction chamber 623 of the device body 620 increases. When the air enters the negative pressure chamber 660 of the breast pump 20 through the air channel 631 of the rear cover 630, the deformable portion 622 can deform toward the direction away from the rear cover 630, at which time the spatial size of the milk suction chamber 623 of the device body 620 decreases.

It is worth mentioning that the specific manner in which the rear cover 630 is installed on the three-way component 610 is not limited in the breast pumping apparatus 20 of the present invention. For example, in this specific example of the breast pumping apparatus 20 of the present invention shown in FIGS. 18 to 22, the top of the rear cover 630 is rotatably mounted to the top of the three-way component 610, and the bottom of the rear cover 630 is detachably mounted to the bottom of the three-way component 610. When the bottom of the rear cover 630 is mounted to the bottom of the three-way component 610, the abutment ring 632 of the rear cover 630 and the rear part of the three-way component 610 can clamp the periphery of the deformable portion 622 to form the negative pressure chamber 660 between the deformable portion 622 and the rear cover 630. When the bottom of the rear cover 630 is separated from the bottom of the three-way component 610, the rear cover 630 can rotate about the connecting shaft between the top of the rear cover 630 and the top of the three-way component 610, so that the rear cover 630 can be flipped. Preferably, the deformable portion 622 has an annular groove 6221, and the abutment ring 632 of the rear cover 630 can be inserted into the annular groove 6221 of the deformable portion 622 to increase the sealing of the negative pressure chamber 660 of the breast pumping apparatus 20.

Specifically, the bottom of the three-way component 610 has at least one slot 614, and the bottom of the rear cover 630 has at least one arm 633. The arm 633 of the rear cover 630 is detachably engaged with the slot 614 of the three-way component 610, so that the bottom of the rear cover 630 is detachably mounted to the bottom of the three-way component 610. Preferably, the bottom of the three-way component 610 has one slot 614 on each of its opposite sides, and the bottom of the rear cover 630 has one arm 633 on each of its opposite sides. The two arms 633 of the rear cover 630 can be engaged with the two slots 614 of the three-way component 610, thereby allowing the bottom of the rear cover 630 to be mounted to the bottom of the three-way component 610.

The duckbill valve 640 and the milk bottle 650 are respectively mounted on the bottom of the three-way component 610, and the milk bottle 650 covers the duckbill valve 640. The duckbill valve 640 is used to selectively open and close the bottom opening 613 of the three-way component 610. In this specific example of the breast pumping apparatus 20 of the present invention shown in FIGS. 18 to 22, the bottom of the three-way component 610 has a valve body mounting arm 615 and a bottle mounting arm 616 surrounding the valve body mounting arm 615. The bottom opening 613 penetrates the valve body mounting arm 615, wherein the duckbill valve 640 is mounted on the valve body mounting arm 615 of the three-way component 610, and the milk bottle 650 is mounted on the bottle mounting arm 616 of the three-way component 610, so that the milk bottle 650 can cover the duckbill valve 640.

When a user uses the breast pumping apparatus 20 of the present invention, on the one hand, the breast closes the opening of the cover portion 621 of the device body 620, so that the breast pumping cavity 623 of the device body 620 forms a closed space; on the other hand, the nipple is extended into the milk suction chamber 623 of the device body 620 and the areola abuts against the areola abutment portion 6211 of the cover portion 621. During the process of discharging air from the negative pressure chamber 660 of the breast pumping apparatus 20 through the air channel 631 of the rear cover 630, the deformable portion 622 deforms toward the rear cover 630. At this time, the duckbill valve 640 closes the bottom opening 613 of the three-way component 610, and the space size of the milk suction chamber 623 of the device body 620 increases, thus creating a negative pressure environment in the milk suction chamber 623. During this process, the areola tends to be pulled toward the milk suction chamber 623 of the device body 620. Since the areola is against the areola abutment portion 6211 of the cover portion 621, the areola abutment portion 6211 of the cover portion 621 prevents the areola from being pulled toward the milk suction chamber 623 of the device body 620, thereby squeezing the areola, discharging milk, and temporarily storing it in the milk suction chamber 623 of the device body 620. When air enters the negative pressure chamber 660 of the breast pumping apparatus 20 through the air channel 631 of the rear cover 630, the deformable portion 622 deforms away from the rear cover 630. At this time, the duckbill valve 640 opens the bottom opening 613 of the three-way component 610, reducing the spatial size of the milk suction chamber 623 of the device body 620. The milk temporarily stored in the milk suction chamber 623 of the device body 620 flows along the guide groove 6213 of the cover portion 621 to the milk discharge channel 6212, and enters the milk bottle 650 after flowing through the milk discharge channel 6212 of the cover portion 621, the bottom opening 613 of the three-way component 610, and the duckbill valve 640. This process is repeated, and the breast pumping apparatus 20 of the present invention can help the user complete the breast pumping action.

Since the cover portion 621 of the device body 620 of the present invention is provided with the guide groove 6213, and the guide groove 6213 is inclinedly extended from the areola abutment portion 6211 to the milk discharge channel 6212, on the one hand, the milk temporarily stored in the milk suction chamber 623 of the device body 620 can be guided by the guide groove 6213 of the cover portion 621 to the milk discharge channel 6212, and subsequently discharged through the milk discharge channel 6212, thus preventing the milk from flowing randomly and causing milk leakage. Regarding the issue of water backflow, on the other hand, the guide groove 6213 of the cover portion 621 is inclinedly extended from the areola abutment portion 6211 to the milk discharge channel 6212. This causes the height of the end of the guide groove 6213 adjacent to the areola abutment portion 6211 to be higher than the height of the end of the guide groove 6213 adjacent to the milk discharge channel 6212. When the user is breastfeeding while leaning against the back of a sofa or bed, the height of the end of the guide groove 6213 of the cover portion 621 adjacent to the areola abutment portion 6211 can still be higher than the height of the end of the guide groove 6213 adjacent to the milk discharge channel 6212. Therefore, the guide groove 6213 of the cover portion 621 can still guide the milk temporarily stored in the milk suction chamber 623 of the device body 620 to the milk discharge channel 6212 to avoid the problem of milk backflow. Thus, the breast pumping apparatus 20 of the present invention allows the user to suckle. Thirdly, the arrangement of the guide groove 6213 of the cover portion 621 increases the capacity of the cover portion 621 for use with the milk suction chamber 6212. The space for temporarily storing milk can prevent milk from flowing back during the pumping process, especially for users with a large milk output, due to the milk level being too high in the milk suction chamber 623 of the main body 620. Fourthly, the areola abutment portion 6211 provided in the cover portion 621 can not only prevent the areola from being pulled toward the milk suction chamber 623 of the main body 620 to squeeze out milk, but also prevent milk from flowing back into the milk suction chamber 623 of the device body 620.

Preferably, the areola abutment portion 6211 of the cover portion 621 is deformable. During milk expression, while the areola abutment portion 6211 of the cover portion 621 prevents the areola from being pulled toward the milk suction chamber 623 of the device body 620 and thus compressing the areola, the areola abutment portion 6211 of the cover portion 621 deforms slightly toward the rear cover 630. Through this process, the breast pumping apparatus 20 can avoid the areola being hard-compressed. In other words, the compression of the areola caused by the areola abutment portion 6211 of the cover portion 621 due to blocking the areola is a gentle compression, avoiding damage to the areola during the milk expression process.

Referring again to FIGS. 18 to 22, the cover portion 621 comprises a cover body 6214 and an inner liner 6215. The milk discharge channel 6212 of the cover portion 621 is formed in the cover body 6214, and the areola abutment portion 6211 and the guide groove 6213 of the cover portion 621 are formed in the inner liner 6215. The inner liner 6215 is installed in the middle section of the cover body 6214, so that the areola abutment portion 6211 of the cover portion 621 is located in the middle section of the cover portion 621. In this way, not only is it beneficial to reduce the manufacturing difficulty and cost of the cover portion 621 and improve the product yield of the cover portion 621, but users can also choose different sizes of the inner liner 6215 according to their own needs. The deformable portion 622 is disposed at the tail end of the cover body 6214. For example, in this specific example of the breast pumping apparatus 20 of the present invention, the deformable portion 622 and the cover body 6214 are integral. The cover body 6214 and the deformable portion 622 can be integrally formed from silicone material by injection molding, or the deformable portion 622 can be integrally bonded to the cover body 6214 by overmolding. This is beneficial to improve the production efficiency of the device body 620 and reduce the assembly steps of the breast pumping apparatus 20.

In other words, in this example of the breast pumping apparatus 20 shown in FIGS. 18 to 22, the inner liner 6215 of the cover portion 621 is detachably installed on the cover body 6214, which allows the inner liner 6215 to be washed and replaced separately. When the inner liner 6215 is installed on the cover body 6214, to ensure that the end of the guide groove 6213 formed in the inner liner 6215 of the cover portion 621 corresponds to the milk discharge channel 6212 formed in the cover body 6214 of the cover portion 621, the cover body 6214 has a body positioning groove 62141, and the inner liner 6215 has an inner liner positioning boss 62151. The inner liner 6215 is installed on the cover body 6214 such that the inner liner positioning boss 62151 of the inner liner 6215 is inserted into the body positioning groove 62141 of the cover body 6214. In other words, during the installation of the inner liner 6215 onto the cover body 6214, as long as the inner liner positioning boss 62151 of the inner liner 6215 is inserted into the body positioning groove 62141 of the cover body 6214, it can be ensured that the end of the guide groove 6213 formed in the inner liner 6213 of the cover body portion 621 corresponds to the milk discharge channel 6212 formed in the cover body 6214 of the cover body portion 621. Furthermore, after the inner liner 6215 is installed onto the cover body 6214, the inner liner positioning boss 62151 of the inner liner 6215 and the body positioning groove 62141 of the cover body 6214 cooperate with each other to prevent the inner liner 6215 from rotating with respect to the cover body 6214, ensuring that the guide groove 6213 of the cover body portion 621 extends to the milk discharge channel 6212.

Preferably, the position of the guide groove 6213 of the cover portion 621 corresponds to the position of the inner liner positioning boss 62151 of the inner liner 6215. In this way, the guide groove 6213 of the cover portion 621 can have a larger tilt angle, that is, the end of the guide groove 6213 of the cover portion 621 adjacent to the areola abutment portion 6211 and the end thereof adjacent to the milk discharge channel 6212 have a larger height difference, which meets the user's sucking needs.

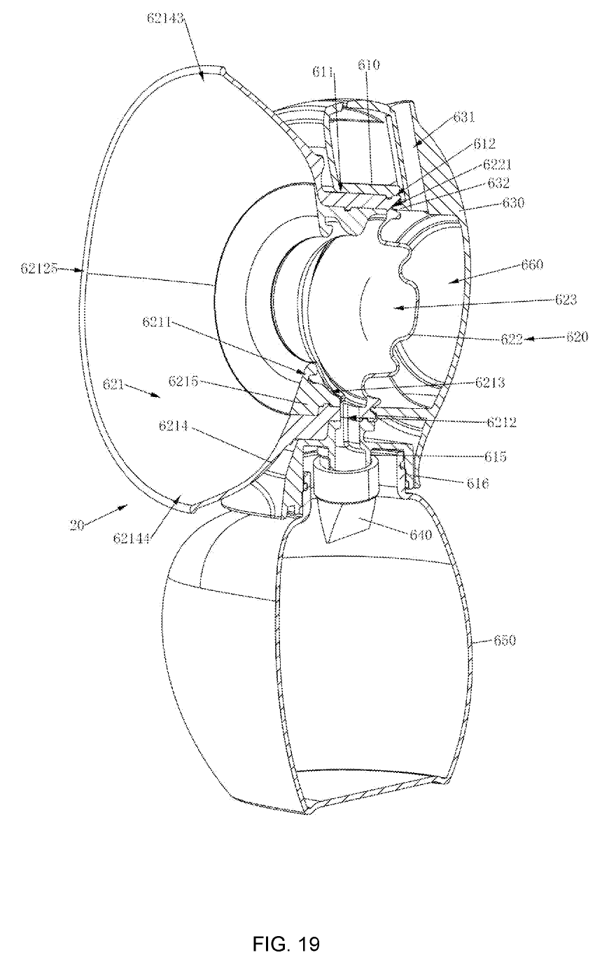

Referring to FIGS. 19 to 22, the inner liner 6215 has a notch 62152, and the guide groove 6213 of the cover portion 621 extends to the notch 62152. After the inner liner 6215 is installed in the middle section of the cover body 6214, the milk discharge channel 6212 of the cover portion 621 corresponds to the notch 62152 of the inner liner 6215, such that the guide groove 6213 of the cover portion 621 extends to the milk discharge channel 6212. By providing the notch 62152 in the inner liner 6215, while ensuring that the inner liner 6215 has a longer length to increase the contact area between the inner liner 6215 and the cover body 6214, the guide groove 6213 of the cover body 621 can have a shorter length. This ensures that the areola abutment portion 6211 of the cover body 621 can be retracted to a position surrounded by the three-way component 610. In this way, the center of gravity of the breast pumping apparatus 20 is closer to the human body, which is beneficial for the breast pump 20 to reliably adhere to the breast when the device body 620 is implemented as a self-adhesive cover.

Additionally, referring to FIGS. 19 to 22, during the process of installing the device body 620 onto the three-way component 610 through the front opening 611, in order to ensure that the milk discharge channel 6212 of the cover portion 621 corresponds to the bottom opening 613 of the three-way component 610, the three-way component 610 has a component positioning groove 617, and the cover body 6214 has a body positioning boss 62142. The cover body 6214 is installed onto the three-way component 610 by inserting the body positioning boss 62142 of the cover body 6214 into the component positioning groove 617 of the three-way component 610. In other words, during the installation of the device body 620 onto the three-way component 610, as long as the body positioning boss 62142 of the cover body 6214 is inserted into the component positioning groove 617 of the three-way component 610, it can be ensured that the milk discharge channel 6212 of the cover body portion 621 corresponds to the bottom opening 613 of the three-way component 610. After the device body 620 is installed onto the three-way component 610, the body positioning boss 62142 of the cover body 6214 and the component positioning groove 617 of the three-way component 610 cooperate with each other to prevent the device body 620 from rotating with respect to the three-way component 610, ensuring that the milk discharge channel 6212 of the cover body portion 621 corresponds to the bottom opening 613 of the three-way component 610.

Referring again to FIGS. 18 to 20, the inner wall of the cover body 6214 is a concave arc surface. This allows the shape of the cover body 6214 to match the shape of the breast, and also allows the opening size of the cover body 6214 to be controlled to a reasonable size. When the breast is contained within the cover body 6214, the areola abuts against the areola abutment portion 6211 of the breast shield portion 621, the inner wall of the cover body 6214 fits tightly against the breast, and the opening edge of the cover body 6214 fastens to the base of the breast, allowing the cover body 6214 to self-adhere to the breast, achieving "naked breastfeeding". In other words, during breastfeeding, the user does not need to hold the breast pumping apparatus 20 by hand or require a bra for support, thus improving the convenience and comfort of using the breast pump.

Furthermore, a top protrusion 62143 is formed at the top of the cover body 6214, a bottom protrusion 62144 is formed at the bottom of the cover body 6214, and a contraction portion 62145 is formed on each of the opposite sides of the cover body 6214. The vertical distance between the top protrusion 62143 and the areola abutment portion 6211 is greater than the vertical distance between the bottom protrusion 62144 and the areola abutment portion 6211, and the vertical distance between the bottom protrusion 62144 and the areola abutment portion 6211 is greater than the vertical distance between the top protrusion 62143 and the areola abutment portion 6211. The vertical distance between the contraction portion 62145 and the areola abutment portion 6211 allows the shape of the cover body 6214 to match the shape of the breast. Furthermore, after the user attaches the cover body 6214 to the breast, the top protrusion 2156 and the bottom protrusion 2157 extend toward the root of the breast from the upper and lower parts, respectively, to increase the contact area between the cover body 6214 and the breast, thereby ensuring that the cover body 6214 can firmly adhere to the breast.

Preferably, the curvature of the bottom of the cover body 6214 is greater than that of the top, so that the shape of the cover body 6214 can better match the shape of the breast.

Referring again to FIGS. 18 to 22, the cover portion 621 further comprises a support ring 6216. The support ring 6216 is protrudingly disposed on the outer wall of the cover body 6214. After the device body 620 is installed on the three-way component 610, the support ring 6216 protrudes toward the three-way component 610 and can abut against the three-way component 610. In this way, during the milk expression process, the support ring 6216 is used to support the lower side of the cover body 6214 and the three-way component 610. The whole formed by the three-way component 610, the rear cover 630, the duckbill valve 640 and the milk bottle 650 will not sag with the increase of its own weight, causing the nipple to lose balance in the milk suction chamber 623 of the device body 620, thereby avoiding milk leakage. Meanwhile, since the support ring 6216 is annular, the support ring 6216 itself can maintain the shape of the cover body 6214 and reduce the deformation range of the cover body 6214, so that no gap will be generated between the breast and the cover body 6214 when the user uses the breast pump 20.

Additionally, the assembly process of the breast pumping apparatus 20 of the present invention shown in FIGS. 18 to 22 can be as follows: First, after aligning the body positioning boss 62142 of the cover body 6214 with the component positioning groove 617 of the three-way component 610, the rear part of the cover body 6214 is pushed into the three-way component 610 through the front opening 611, and the deformable portion 622 extends out of the three-way component 610 through the rear opening 612, and the milk discharge channel 6212 of the cover portion 621 is communicated to the bottom opening 613 of the three-way component 610. Next, flip the rear cover 630 so that the arm 633 of the rear cover 630 extends into the slot 614 of the three-way component 610, so that the bottom of the rear cover 630 is installed at the bottom of the three-way component 610, wherein the abutment ring 632 of the rear cover 630 and the rear part of the three-way component 610 clamp the periphery of the deformable portion 622 to form the negative pressure chamber 660 between the deformable portion 622 and the rear cover 630. Third, first install the duckbill valve 640 on the valve body mounting arm 615 of the three-way component 610, and then install the milk bottle 650 on the milk bottle mounting arm 616 of the three-way component 610, wherein the milk bottle 650 covers the duckbill valve 640. Fourth, after aligning the inner liner positioning boss 62151 of the inner liner 6215 with the body positioning groove 62141 of the cover body 6214, the inner liner 6215 is pushed into the cover body 6214 to install the inner liner 6215 onto the cover body 6214, wherein the milk discharge channel 6212 of the cover body portion 621 corresponds to the notch 62152 of the inner liner 6215. Through these steps, the breast pumping apparatus 20 can be assembled. It is understood that the order of the above steps can be changed as needed, and the breast pumping apparatus 20 of the present invention is not limited in this respect.

Additionally, it should be noted that the inner liner 6215 of the present invention can be disposed in the horn cover of the prior art (for example, the horn cover of the breast pump disclosed in the invention patent application number CN118873774A ), so that the horn cover of the prior art has the function of guiding milk flow and preventing milk backflow.

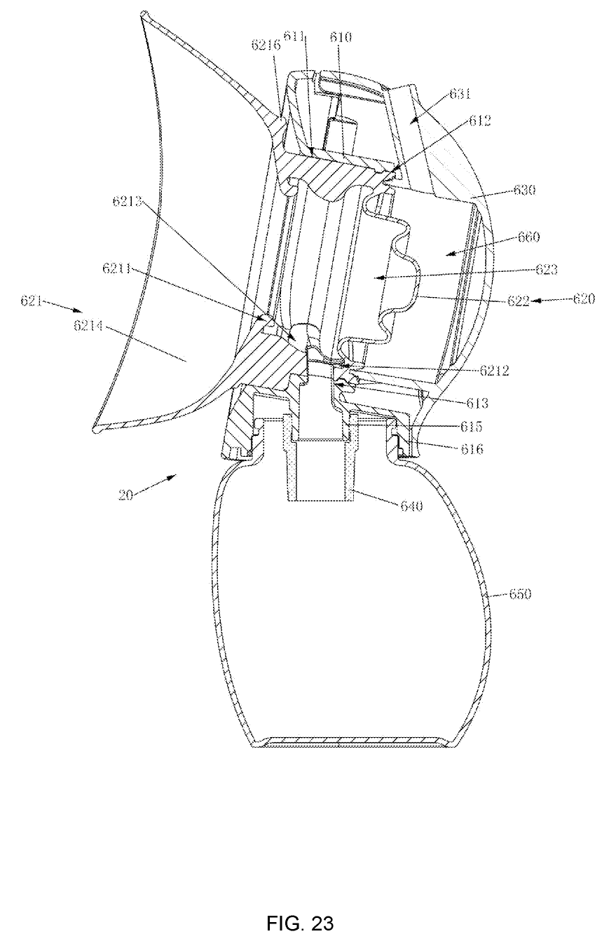

FIG. 23 shows a modified example of the breast pumping apparatus 20 according to the above described preferred embodiment of the present invention. Unlike the breast pumping apparatus 20 shown in FIGS. 18 to 22, in this specific example of the breast pumping apparatus 20 shown in FIG. 23, the cover portion 621 does not have the inner liner 6215, and the areola abutment portion 6211, the milk discharge channel 6212, and the guide groove 6213 of the cover portion 621 are all formed on the cover body 6214.

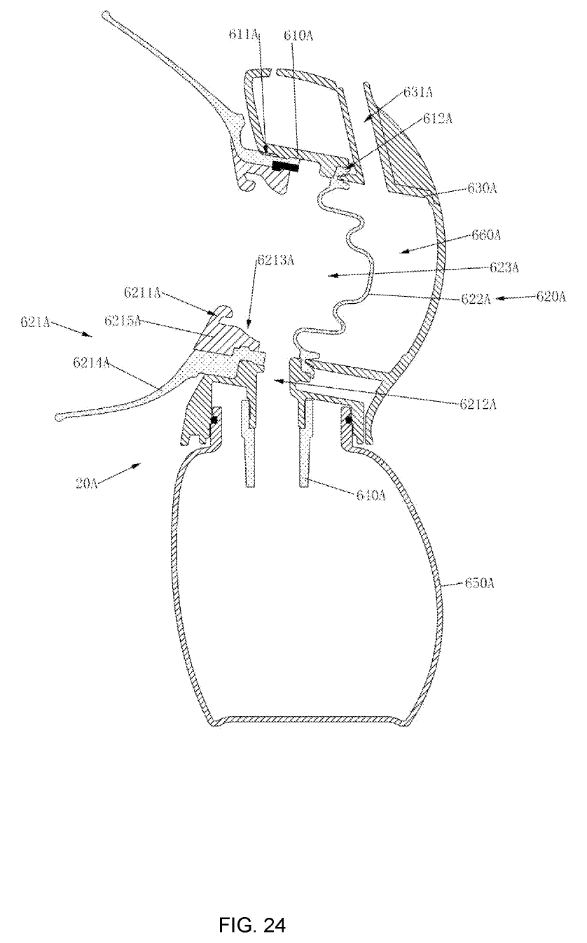

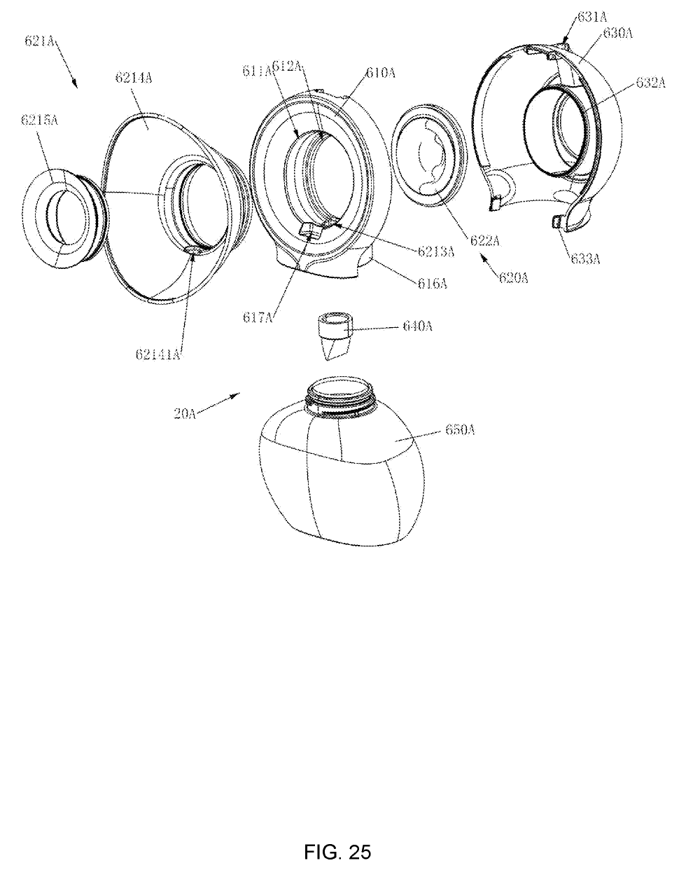

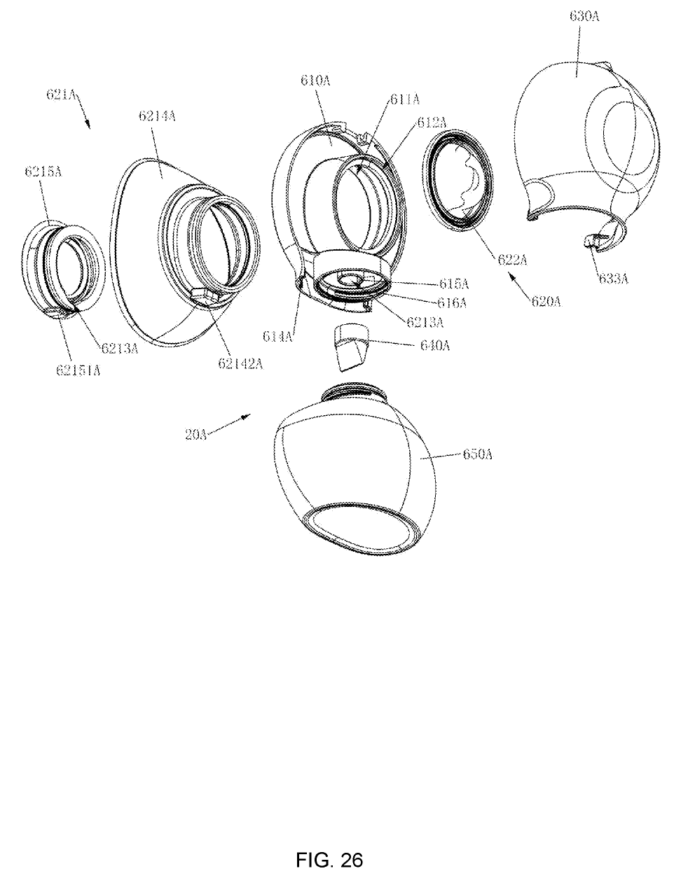

FIGS. 24 to 26 show a breast pumping apparatus 20A according to another preferred embodiment. The breast pumping apparatus 20A comprises a three-way component 610A, a device body 620A, a rear cover 630A, a duckbill valve 640A, and a milk bottle 650A. The breast pumping apparatus 20A has a milk suction chamber 623A and a negative pressure chamber 660A.

The three-way component 610A has a front opening 611A, a rear opening 612A, and a milk discharge channel 6212A.

The device body 620A comprises a cover portion 621A and a deformable portion 622A. The cover portion 621A has an areola abutment portion 6211A and a guide groove 6213A. The areola abutment portion 6211A is located in the middle section of the cover portion 621A, and the guide groove 6213A extends from the areola abutment portion 6211A to the tail end of the cover portion 621A. When the cover portion 621A is installed on the three-way component 610A via the front opening 611A, the guide groove 6213A of the cover portion 621A extends to the milk discharge channel 6212A of the three-way component 610A. The deformable portion 622A is installed on the three-way component 610A and is used to close the rear opening 612A of the three-way component 610A. Thus, the breast pumping apparatus 20A forms the milk suction chamber 623A between the cover portion 621A, the three-way component 610A, and the deformable portion 622A. It is understood that the milk discharge channel 6212A and the milk suction chamber 623A of the three-way component 610A are connected.

The rear cover 630A has an air channel 631A. The rear cover 630A is installed on the three-way component 610A. The periphery of the deformable portion 622A is clamped between the three-way component 610A and the rear cover 630A, so that the deformable portion 622A is installed on the three-way component 610A and is used to close the rear opening 612A of the three-way component 610A. The milk suction chamber 623A of the milk suction device 20A is formed between the deformable portion 622A and the rear cover 630A, and the air channel 631A of the rear cover 630A is communicated to the negative pressure chamber 660A of the milk suction device 20A. It is understood that when the air in the negative pressure chamber 660A of the breast pump 20A is discharged through the air channel 631A of the rear cover 630A, the deformable portion 622A can deform toward the direction closer to the rear cover 630A. When the air enters the negative pressure chamber 660A of the breast pump 20A through the air channel 631A of the rear cover 630A, the deformable portion 622A can deform toward the direction away from the rear cover 630A.

It is worth mentioning that the specific manner in which the rear cover 630A is installed on the three-way component 610A is not limited in the breast pumping apparatus 20A of the present invention. For example, in this specific example of the breast pumping apparatus 20A of the present invention shown in FIGS. 24 to 26, the top of the rear cover 630A is rotatably mounted to the top of the three-way component 610A, and the bottom of the rear cover 630A is detachably mounted to the bottom of the three-way component 610A. When the bottom of the rear cover 630A is mounted to the bottom of the three-way component 610A, the abutment ring 632A of the rear cover 630A and the rear part of the three-way component 610A can clamp the periphery of the deformable portion 622A to form the negative pressure chamber 660A between the deformable portion 622A and the rear cover 630A. When the bottom of the rear cover 630A is separated from the bottom of the three-way component 610A, the rear cover 630A can rotate about the connecting shaft between the top of the rear cover 630A and the top of the three-way component 610A, so that the rear cover 630A can be flipped.

Specifically, the bottom of the three-way component 610A has at least one slot 614A, and the bottom of the rear cover 630A has at least one arm 633A. The arm 633A of the rear cover 630A is detachably engaged with the slot 614A of the three-way component 610A, so that the bottom of the rear cover 630A is detachably mounted to the bottom of the three-way component 610A. Preferably, the bottom of the three-way component 610A has one slot 614A on each of its opposite sides, and the bottom of the rear cover 630A has one arm 633A on each of its opposite sides. The two arms 633A of the rear cover 630A can be engaged into the two slots 614A of the three-way component 610A, thereby allowing the bottom of the rear cover 630A to be mounted to the bottom of the three-way component 610A.

The duckbill valve 640A and the milk bottle 650A are respectively installed at the bottom of the three-way component 610A, and the milk bottle 650A covers the duckbill valve 640A. The duckbill valve 640A is used to selectively open and close the milk discharge channel 6212A of the three-way component 610A. In this specific example of the breast pumping apparatus 20A of the present invention shown in FIGS. 24 to 26, the bottom of the three-way component 610A has a valve body mounting arm 615A and a bottle mounting arm 616A surrounding the valve body mounting arm 615A. The milk discharge channel 6212A passes through the valve body mounting arm 615A. The duckbill valve 640A is mounted on the valve body mounting arm 615A of the three-way component 610A, and the milk bottle 650A is mounted on the bottle mounting arm 616A of the three-way component 610A, so that the milk bottle 650A can cover the duckbill valve 640A.

When a user uses the breast pumping apparatus 20A of the present invention, on the one hand, the breast closes the opening of the cover portion 621A of the device body 620A, forming a closed space in the milk suction chamber 623A of the breast pumping apparatus 20A; on the other hand, the nipple extends into the milk suction chamber 623A of the breast pumping apparatus 20A, and the areola touches the areola abutment portion 6211A of the cover portion 621A. During the process of expelling air from the negative pressure chamber 660A of the breast pumping apparatus 20A through the air channel 631A of the rear cover 630A, the deformable portion 622A deforms towards the rear cover 630A. At this time, the duckbill valve 640A closes the milk discharge channel 6212A of the three-way component 610A, increasing the space size of the milk suction chamber 623A of the breast pumping apparatus 20A and creating a negative pressure in the milk suction chamber 623A. In this process, the areola tends to be pulled toward the milk suction chamber 623A of the milk suction device 20A. Since the areola is against the areola abutment portion 6211A of the cover portion 621A, the areola abutment portion 6211A of the cover portion 621A prevents the areola from being pulled toward the milk suction chamber 623A of the milk suction device 20A, thereby squeezing the areola, expelling milk, and temporarily storing it in the milk suction chamber 623A of the milk suction device 20A. When air enters the negative pressure chamber 660A of the breast pumping apparatus 20A through the air channel 631A of the rear cover 630A, the deformable portion 622A deforms away from the rear cover 630A. At this time, the duckbill valve 640A opens the milk discharge channel 6212A of the three-way component 610A, reducing the space size of the milk suction chamber 623A of the breast pumping apparatus 20A. The milk temporarily stored in the milk suction chamber 623A of the breast pumping apparatus 20A flows along the guide groove 6213A of the cover portion 621A to the milk discharge channel 6212A of the three-way component 610A, and then enters the milk bottle 650A after flowing through the milk discharge channel 6212A and the duckbill valve 640A. This process repeats, and the breast pumping apparatus 20A of the present invention can help the user complete the breast pumping action.

Since the cover portion 621A of the device body 620A of the present invention is provided with the guide groove 6213A, and the guide groove 6213A is inclinedly extended from the areola abutment portion 6211A to the tail end of the cover portion 621A, on the one hand, the milk temporarily stored in the milk suction chamber 623A of the milk suction device 20A can be guided by the guide groove 6213A of the cover portion 621A to the milk discharge channel 6212A of the three-way component 610A, and subsequently discharged through the milk discharge channel 6212A, thus preventing the milk from flowing randomly and causing problems. Regarding the issue of milk backflow, on the other hand, the guide groove 6213A of the cover portion 621A is inclinedly extended from the areola abutment portion 6211A to the tail end of the cover portion 621A and extends to the milk discharge channel 6212A after the cover portion 621A is installed on the three-way component 610A. This makes the height of the end of the guide groove 6213A adjacent to the areola abutment portion 6211A higher than the height of the end of the guide groove 6213A adjacent to the milk discharge channel 6212A. When the user is breastfeeding while leaning against the back of a sofa or bed, the height of the end of the guide groove 6213A adjacent to the areola abutment portion 6211A can still be higher than the height of the end of the guide groove 6213A adjacent to the milk discharge channel 6212A. Therefore, the guide groove 6213A of the cover portion 621A can still guide the milk temporarily stored in the milk suction chamber 623A of the milk suction device 20A to the milk discharge channel 6212A, thus avoiding the problem of milk backflow. This allows the breast pumping apparatus 20A of the present invention to allow the user to suckle. Thirdly, the addition of the guide groove 6213A of the cover portion 621A increases the space for temporary milk storage in the cover portion 621A. For a user with a large milk output, this prevents milk from flowing back during the pumping process due to the milk level in the milk suction chamber 623A of the breast pumping apparatus 20A being too high. Fourthly, the areola abutment portion 6211A in the cover portion 621A not only prevents the areola from being pulled towards the milk suction chamber 623A of the breast pumping apparatus 20A to squeeze out milk, but also prevents milk from flowing back into the milk suction chamber 623A of the breast pumping apparatus 20A.

Referring again to FIGS. 24 to 26, the cover portion 621A comprises a cover body 6214A and an inner liner 6215A. The areola abutment portion 6211A and the guide groove 6213A of the cover portion 621A are formed in the inner liner 6215A. The inner liner 6215A is installed in the middle section of the cover body 6214A, so that the areola abutment portion 6211A of the cover portion 621A is located in the middle section of the cover portion 621A. In this way, not only is it beneficial to reduce the manufacturing difficulty and cost of the cover portion 621A and improve the product yield of the cover portion 621A, but users can also choose different sizes of the inner liner 6215A according to their own needs.

In other words, in this example of the breast pumping apparatus 20A shown in FIGS. 24 to 26, the inner liner 6215A of the cover portion 621A is detachably installed on the cover body 6214A, which allows the inner liner 6215A to be washed and replaced separately. When the inner liner 6215A is installed on the cover body 6214A, to ensure that the shape of the edge of the cover portion 621A corresponds to the position of the guide groove 6213A formed in the inner liner 6215A, the cover body 6214A has a body positioning groove 62141A, and the inner liner 6215A has an inner liner positioning boss 62151A. The inner liner 6215A is installed on the cover body 6214A by inserting the inner liner positioning boss 62151A into the body positioning groove 62141A of the cover body 6214A. In other words, during the installation of the inner liner 6215A onto the cover body 6214A, as long as the inner liner positioning boss 62151A of the inner liner 6215A is inserted into the body positioning groove 62141A of the cover body 6214A, it can be ensured that the shape of the edge of the cover portion 621A corresponds to the position of the guide groove 6213A formed in the inner liner 6215A. Furthermore, after the inner liner 6215A is installed onto the cover body 6214A, the inner liner positioning boss 62151A of the inner liner 6215A and the body positioning groove 62141A of the cover body 6214A cooperate with each other to prevent the inner liner 6215A from rotating with respect to the cover body 6214A, ensuring that the shape of the edge of the cover portion 621A and the position of the guide groove 6213A formed in the inner liner 6215A always correspond.

Preferably, the position of the guide groove 6213A of the cover portion 621A corresponds to the position of the inner liner positioning boss 62151A of the inner liner 6215A. In this way, the guide groove 6213A of the cover portion 621A can have a larger tilt angle, that is, the end of the flow guide groove 6213A of the cover portion 621A adjacent to the areola abutment portion 6211A and the end adjacent to the milk discharge channel 6212A have a larger height difference, which meets the user's need for breastfeeding.

Additionally, referring to FIGS. 24 to 26, during the process of installing the device body 620A onto the three-way component 610A through the front opening 611A, in order to ensure that the flow guide groove 6213A of the cover portion 621A corresponds to the milk discharge channel 6212A of the three-way component 610A, the three-way component 610A has a component positioning groove 617A, and the cover body 6214A has a body positioning boss 62142A. The cover body 6214A is installed onto the three-way component 610A by inserting the body positioning boss 62142A of the cover body 6214A into the component positioning groove 617A of the three-way component 610A. In other words, during the installation of the device body 620A onto the three-way component 610A, as long as the body positioning boss 62142A of the cover body 6214A is inserted into the component positioning groove 617A of the three-way component 610A, it can be ensured that the flow guide groove 6213A of the cover body portion 621A corresponds to the milk discharge channel 6212A of the three-way component 610A. Furthermore, after the device body 620A is installed onto the three-way component 610A, the body positioning boss 62142A of the cover body 6214A and the component positioning groove 617A of the three-way component 610A cooperate with each other to prevent the device body 620A from rotating with respect to the three-way component 610A, thus ensuring that the flow guide groove 6213A of the cover body portion 621A corresponds to the milk discharge channel 6212A of the three-way component 610A.

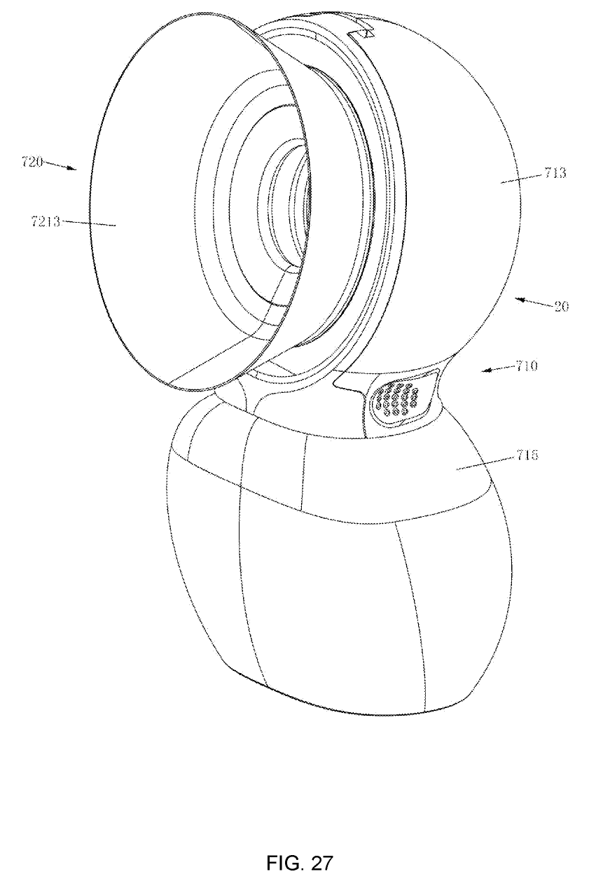

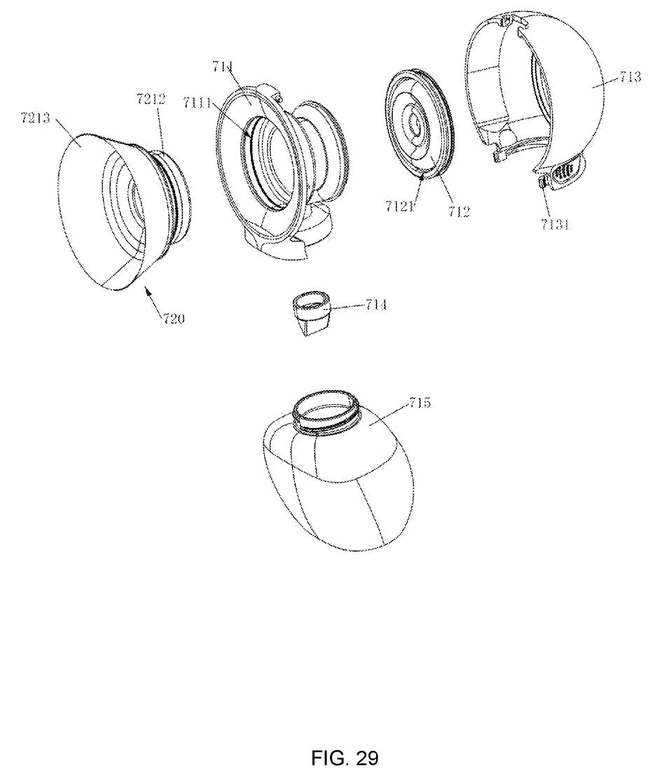

FIGS. 27 to 30 show a breast pumping apparatus 20 according to another preferred embodiment, wherein the breast pumping apparatus 20 comprises a device body 710 and a horn cover 720 mounted on the device body 710.

Specifically, the horn cover 720 comprises a self-adhesive cover 721 and a support ring 722, the support ring 722 being protrudingly disposed on the outer wall of the self-adhesive cover 721. Preferably, the self-adhesive cover 721 and the support ring 722 are integral, for example, the self-adhesive cover 721 and the support ring 722 can be integrally formed from silicone material by injection molding. The self-adhesive cover 721 can be installed on the device body 710. After the self-adhesive cover 721 is installed on the device body 710, an active space 7100 is formed between the self-adhesive cover 721 and the device body 710. The support ring 722 is located in the active space 7100 and can abut against the device body 710. The support ring 722 supports the lower side of the self-adhesive cover 721 and the device body 710. In this way, during the milk pumping process, the device body 710 will not sag due to its own weight, causing the nipple to lose balance in the milk suction chamber 7101 of the device body 710. This prevents milk leakage and ensures the suction of the cover 720, so that the breast pump is reliably attached to the breast.

Additionally, it is understood that the support ring 722 supports the self-adhesive cover 721 and the device body 710 on the lower side of the self-adhesive cover 721. In this way, the deformation range of the horn cover 720 can be greatly reduced to ensure the adhesion between the horn cover 720 and the breast, so that the breast pump can be reliably adhered to the breast.

It is worth mentioning that, in one example of the breast pump of the present invention, after the self-adhesive cover 721 is installed on the device body 710, the support ring 722 can directly abut against the device body 710. In another example of the breast pump of the present invention, after the self-adhesive cover 721 is installed on the device body 710, there may be a gap between the support ring 722 and the device body 710. When the user uses the breast pump to express milk, after the self-adhesive cover 721 is slightly deformed, the support ring 722 can abut against the device body 710, so that the support ring 722 supports the lower side of the self-adhesive cover 721 and the device body 710.

Preferably, the thickness of the portion of the self-adhesive cover 721 located outside the support ring 722 gradually decreases from the support ring 722 outwards.

Referring again to FIGS. 27 to 30, the device body 710 further comprises a milk discharge channel 7102 and an assembly opening 7103, both of which are connected to the milk suction chamber 7101. The self-adhesive cover 721 is installed on the main body 710 at the assembly opening 7103. When the user uses the breast pump, the self-adhesive cover 721 adheres to the breast, and the nipple extends into the milk suction chamber 7101 of the main body 710. As the pressure within the milk suction chamber 7101 of the main body 710 changes rhythmically, milk is drawn in and discharged from the milk suction chamber 7101 through the milk discharge channel 7102. Since the support ring 722 supports the self-adhesive cover 721 and the device body 710 on the lower side of the self-adhesive cover 721, the relative position of the nipple and the device body 710 can remain stable throughout the milk pumping process. This prevents the nipple from losing its balance in the milk suction chamber 7101 of the device body 710, thus preventing milk leakage and ensuring the suction properties of the horn cover 720, so that the breast pump is reliably attached to the breast.

Specifically, the device body 710 comprises a three-way component 711, a deformable negative pressure forming part 712, a housing 713, a one-way valve 714, and a milk bottle 715. The three-way component 711 has a front opening 7111, a rear opening 7112, and a bottom opening 7113. The front opening 7111 of the three-way component 711 forms the assembly opening 7103 of the device body 710, and the bottom opening 7113 of the three-way component 711 forms the milk discharge channel 7102 of the device body 710. The housing 713 is mounted on the three-way component 711, and the negative pressure forming part 712 is clamped within the three-way component. Between the rear part of component 711 and the housing 713, and the negative pressure forming part 712 closes the rear opening 7112 of the three-way component 711, the device body 710 forms the milk suction chamber 7101 between the three-way component 711 and the negative pressure forming part 712. When the negative pressure forming part 712 deforms, the magnitude of the negative pressure in the milk suction chamber 7101 changes. The one-way valve 714 and the milk bottle 715 are respectively installed at the bottom of the three-way component 711, and the one-way valve 714 selectively opens and closes the bottom opening 7113 of the three-way component 711. The milk bottle 715 covers the one-way valve 714. Specifically, when the negative pressure in the milk suction chamber 7101 of the device body 710 increases, the one-way valve 714 closes the bottom opening 7113 of the three-way component 711, that is, the milk discharge channel 7102 of the device body 710 is closed. Correspondingly, when the negative pressure in the milk suction chamber 7101 of the device body 710 decreases, the one-way valve 714 opens the bottom opening 7113 of the three-way component 711, that is, the milk discharge channel 7102 of the device body 710 is opened.

Furthermore, the top of the housing 713 is rotatably mounted to the upper side of the three-way component 711 at the rear, and the bottom of the housing 713 is detachably mounted to the lower side of the three-way component 711 at the rear. After the bottom of the housing 713 is separated from the lower side of the three-way component 711, the housing 713 can be flipped up, allowing the negative pressure forming part 712 to be disassembled for cleaning. After the bottom of the housing 713 is installed on the lower side of the three-way component 711, the three-way component 711 and the housing 713 clamp the negative pressure forming part 712, so that the negative pressure forming part 712 closes the rear opening 7112 of the three-way component 711, allowing the device body 710 to form the milk suction chamber 7101 between the three-way component 711 and the negative pressure forming part 712.

It is worth mentioning that the specific structure in which the bottom of the housing 713 and the lower side of the three-way component 711 are detachably installed is not limited in the breast pump of the present invention. For example, in this specific example of the breast pumping apparatus 20 of the present invention shown in FIGS. 27 to 30, the bottom of the housing 713 has a buckle 7131 on each of its opposite sides, and the lower side of the three-way component 711 has a slot 7114 on each of its opposite sides. The two buckles 7131 of the housing 713 can be respectively engaged with the two slots 7114 of the three-way component 711. When the two buckles 7131 of the housing 713 are respectively engaged with the two slots 7114 of the three-way component 711, the bottom of the housing 713 and the lower side of the three-way component 711 are installed. When the two buckles 7131 of the housing 713 are respectively disengaged from the two slots 7114 of the three-way component 711, the bottom of the housing 713 and the lower side of the three-way component 711 are separated.

Preferably, the negative pressure forming part 712 has an annular groove 7121, and the rear part of the three-way component 711 is inserted into the annular groove 7121 of the negative pressure forming part 712. In this way, when the bottom of the housing 713 is installed on the lower side of the three-way component 711 and the negative pressure forming part 712 is clamped between the housing 713 and the three-way component 711, the negative pressure forming part 712 can reliably seal the rear opening 7112 of the three-way component 711. Furthermore, when the negative pressure forming part 712 is installed between the three-way component 711 and the housing 713, after the rear part of the three-way component 711 is inserted into the annular groove 7121 of the negative pressure forming part 712, the negative pressure forming part 712 can be pre-installed on the three-way component 711, so that when the housing 713 is flipped over and the bottom of the housing 713 and the lower side of the three-way component 711 are allowed to be installed, the negative pressure forming part 712 can be automatically clamped between the three-way component 711 and the housing 713.

Referring again to FIGS. 27 to 30, the self-adhesive cover 721 of the horn cover 720 further comprises a shoulder ring 7211, a mounting ring 7212, and a deformable horn cover element 7213. The mounting ring 7212 is extended to one side from the inner edge of the shoulder ring 7211, and the horn cover element 7213 extends in the opposite direction from the outer edge of the shoulder ring 7211. The support ring 722 is disposed at the connection position between the shoulder ring 7211 and the horn cover element 7213. The mounting ring 7212 of the self-adhesive cover 721 can extend through the front opening 7111 of the three-way component 711 into the interior of the three-way component 711, thereby allowing the self-adhesive cover 721 to be mounted on the device body 710 at the assembly opening 7103. The support ring 722 supports the self-adhesive cover 721 and the device body 710 on the lower side of the self-adhesive cover 721. This significantly reduces the deformation range of the horn cover 720, ensuring the adhesion between the horn cover 720 and the breast, allowing the breast pump to be reliably attached to the breast. Optionally, in other examples of the breast pump of the present invention, the support ring 722 can be disposed on the shoulder ring 7211 of the self-adhesive cover 721.

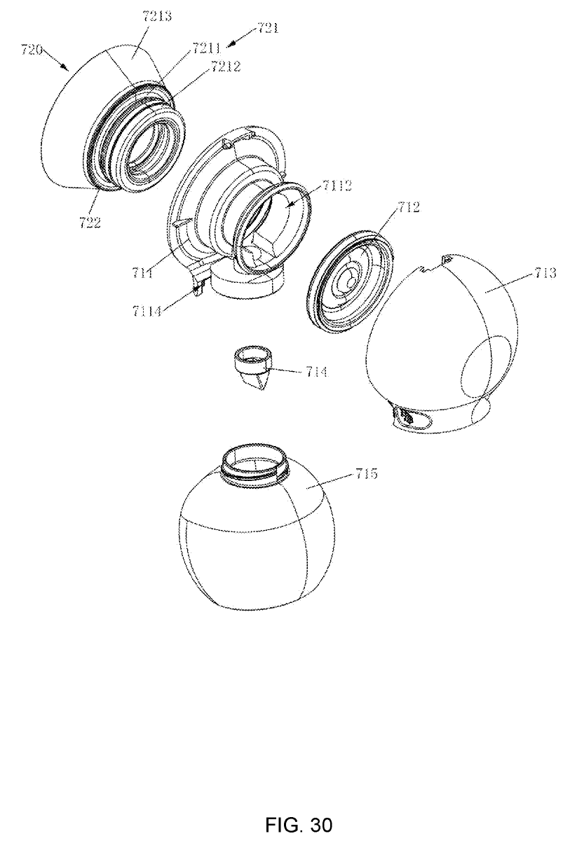

Preferably, referring to FIG. 30, the support ring 722 surrounds the shoulder ring 7211 of the self-adhesive cover 721. Thus, the support ring 722, acting as a support frame, maintains the shape of the horn cover 720, reducing the deformation range of the horn cover 720 and ensuring the adhesion between the horn cover 720 and the breast. In other words, by making the support ring 722 circular, on the one hand, the support ring 722 can support the self-adhesive cover 721 and the device body 710 on the lower side of the self-adhesive cover 721, preventing the device body 710 from sagging due to its increased weight during milk expression, thus preventing the nipple from becoming unbalanced within the milk suction chamber 7101 of the device body 710. This prevents milk leakage and ensures the adhesion of the horn cover 720 and the reliable adhesion of the breast pump to the breast. On the other hand, the support ring 722 itself maintains the shape of the horn cover 720, reducing the deformation range of the horn cover 720 and ensuring the adhesion between the horn cover 720 and the breast.

Referring again to FIGS. 27 to 30, the self-adhesive cover 721 comprises a blocking portion 7214, which further comprises an installing ring 72141 and a deformable blocking ring 72142. The blocking ring 72142 is extended inwardly from one end of the installing ring 72141, wherein the installing ring 72141 is mounted to the mounting ring 7212. For example, based on the frictional force generated between the installing ring 72141 and the mounting ring 7212, the installing ring 72141 can be mounted to the mounting ring 7212. The blocking ring 72142 is held at the connection position between the shoulder ring 7211 and the mounting ring 7212, and the blocking ring 72142 is extended inwardly at the connection position between the shoulder ring 7211 and the mounting ring 7212. When a user uses the breast pumping apparatus, the nipple is extended into the milk suction chamber 7101 of the device body 710 through the central through hole of the blocking ring 72142 of the blocking portion 7214. The areola is blocked by the blocking ring 72142. When the space of the milk suction chamber 7101 of the device body 710 decreases, resulting in an increase in the negative pressure inside the milk suction chamber 7101, the nipple is pulled towards the milk suction chamber 7101 of the device body 710. The areola is squeezed due to being blocked by the blocking ring 72142, so that milk can be expressed and temporarily stored in the milk suction chamber 7101 of the device body 710. When the space of the milk suction chamber 7101 of the device body 710 increases, resulting in a decrease in the negative pressure inside the milk suction chamber 7101, the one-way valve 714 opens the milk discharge channel 7102 of the device body 710, allowing milk to be discharged into the milk bottle 715. In other words, the blocking ring 72142 of the blocking portion 7214 of the breast pump of the present invention can squeeze out milk, thereby improving the user's comfort and milk expression efficiency when using the breast pump. Furthermore, since the blocking ring 72142 is deformable, as the nipple is pulled toward the milk suction chamber 7101 of the device body 710, the blocking ring 72142 will deform toward the milk suction chamber 7101 of the device body 710 while blocking the areola. That is, the blocking ring 72142 provides flexible blocking, thereby improving the user's comfort when using the breast pump.

Preferably, the end of the blocking ring 72142 is extended curved toward the milk suction chamber 7101 of the device body 710, so that the horn cover 720 can prevent the nipple from contacting the end of the blocking ring 72142, thereby protecting the nipple.

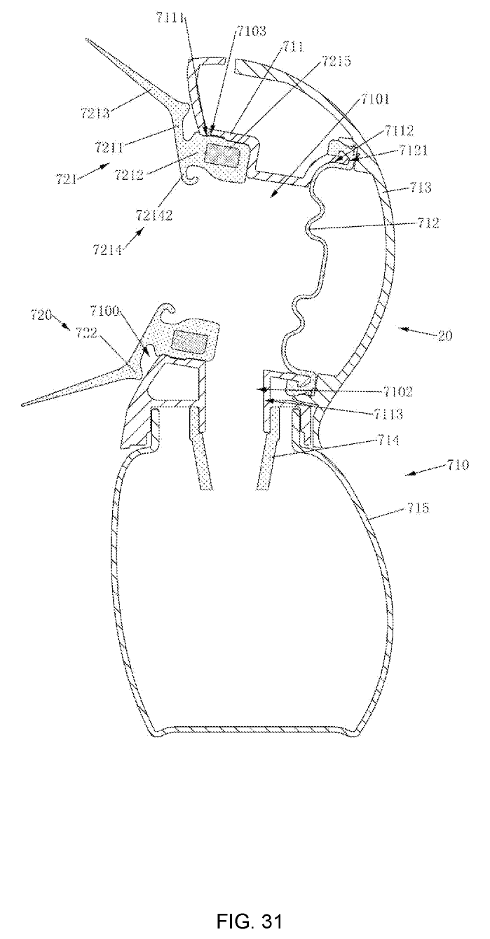

Optionally, in this specific example of the breast pumping apparatus 20 of the present invention shown in FIG. 31, the blocking ring 72142 may be extended inwardly integrally from the connection position of the shoulder ring 7211 and the mounting ring 7212. That is, the self-adhesive cover 721 and the support ring 722 of the horn cover 720 are an integral structure; for example, the horn cover 720 can be made of silicone material into an integral structure by injection molding.

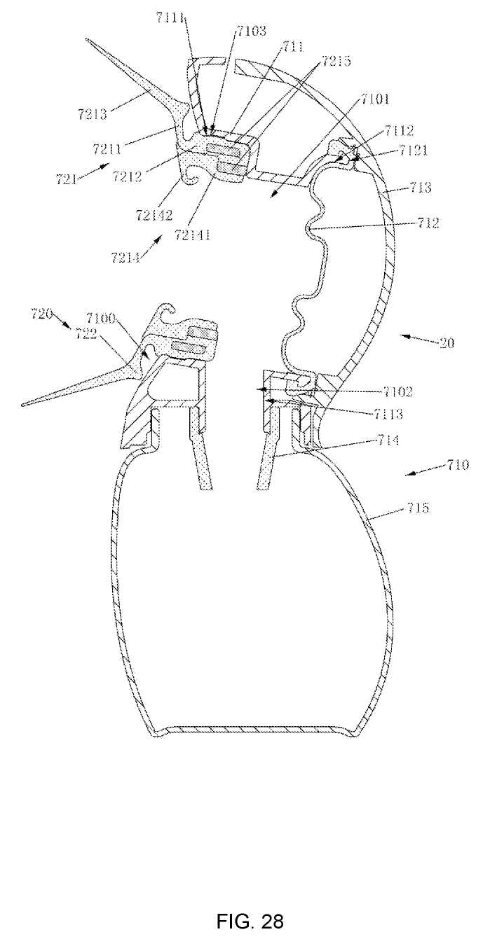

Preferably, referring to FIG. 28, the self-adhesive cover 721 comprises two annular frames 7215. One annular frame 7215 is disposed on the mounting ring 7212 to maintain the shape of the mounting ring 7212, and the other annular frame 7215 is disposed on the installing ring 72141 to maintain the shape of the installing ring 72141, so that the installing ring 72141 can be stably mounted on the mounting ring 7212.

It is worth mentioning that the specific manner in which the annular frame 7215 is set on the mounting ring 7212 is not limited in the breast pumping apparatus 20 of the present invention. For example, an insert injection molding process can embed the annular frame 7215 inside the mounting ring 7212 to set the annular frame 7215 on the mounting ring 7212. Correspondingly, the specific manner in which the annular frame 7215 is set on the installing ring 72141 is not limited in the breast pumping apparatus 20 of the present invention. For example, an overmolding process can fit the installing ring 72141 onto the outside of the installing ring 72141.

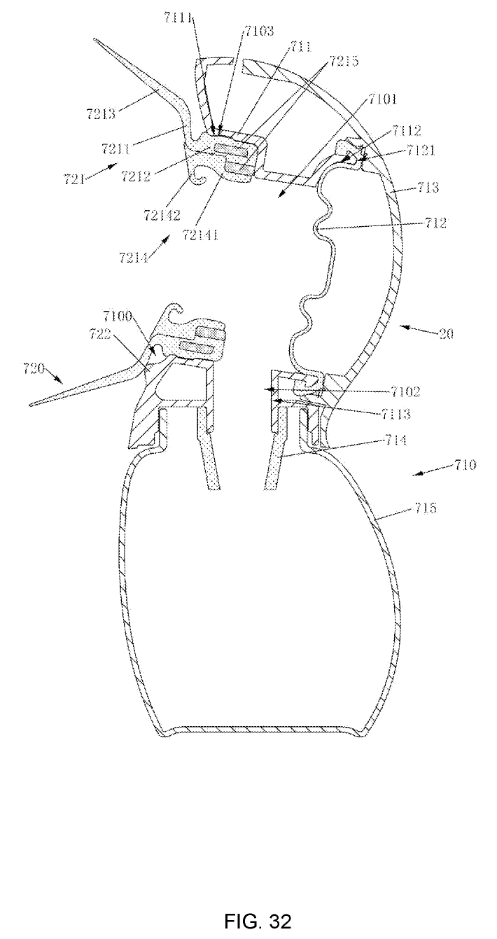

FIG. 32 shows a modified example of the breast pump according to the present invention. Unlike the breast pumps shown in FIGS. 27 to 30, in this specific example of the breast pumping apparatus 20 shown in FIG. 32, the support ring 722 is disposed on the device body 710. When the self-adhesive cover 721 is installed on the device body 710 and the active space 7100 is formed between the self-adhesive cover 721 and the device body 710, the support ring 722 supports the self-adhesive cover 721 on the lower side. Thus, during the breast pumping process, the device body 710 will not sag due to its own weight, causing the nipple to lose balance in the milk suction chamber 7101 of the device body 710. This prevents milk leakage and ensures the suction of the horn cover 720 and the reliable suction of the breast pump on the breast. In this specific example of the breast pumping apparatus 20 shown in FIG. 32, the annular frame 7215 is disposed on the mounting ring 7212 to maintain the shape of the mounting ring 7212. For example, the annular frame 7215 may be embedded inside the mounting ring 7212.

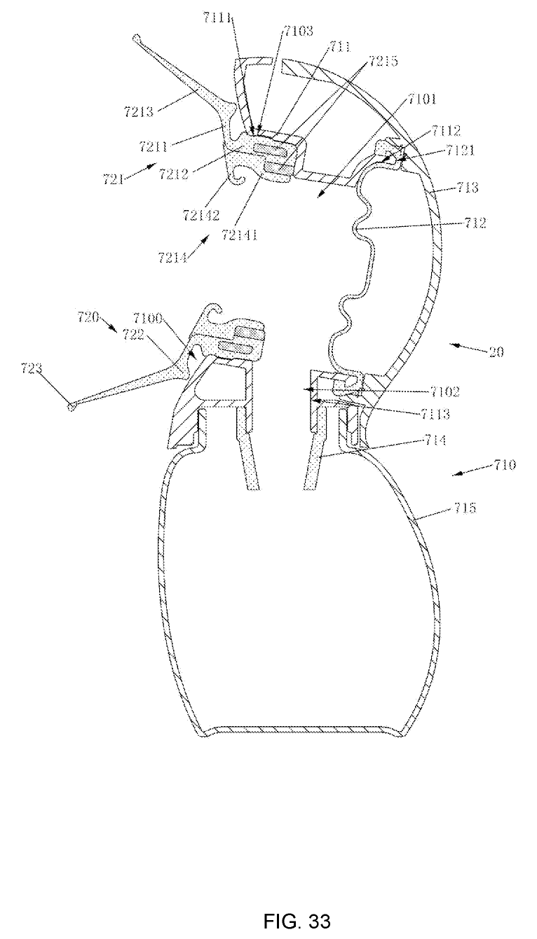

FIG. 33 shows a modified example of the breast pump according to the present invention. Unlike the breast pumps shown in FIGS. 27 to 30, in this specific example of the breast pumping apparatus 20 shown in FIG. 33, the horn cover 720 further comprises a tapered portion 723. The tapered portion 723 is disposed at the periphery of the self-adhesive cover 721 at the external opening, and the thickness of the tapered portion 723 is greater than the thickness of the periphery of the self-adhesive cover 721 at the external opening. In this way, on the one hand, the tapered portion 723 can prevent the self-adhesive cover 721 from breaking due to its thin periphery at the external opening; on the other hand, the tapered portion 723 can increase the strength of the periphery of the self-adhesive cover 721 at the external opening, which is beneficial to maintaining the periphery shape of the self-adhesive cover 721 at the external opening, thereby enabling the self-adhesive cover 721 to reliably adhere to the breast.

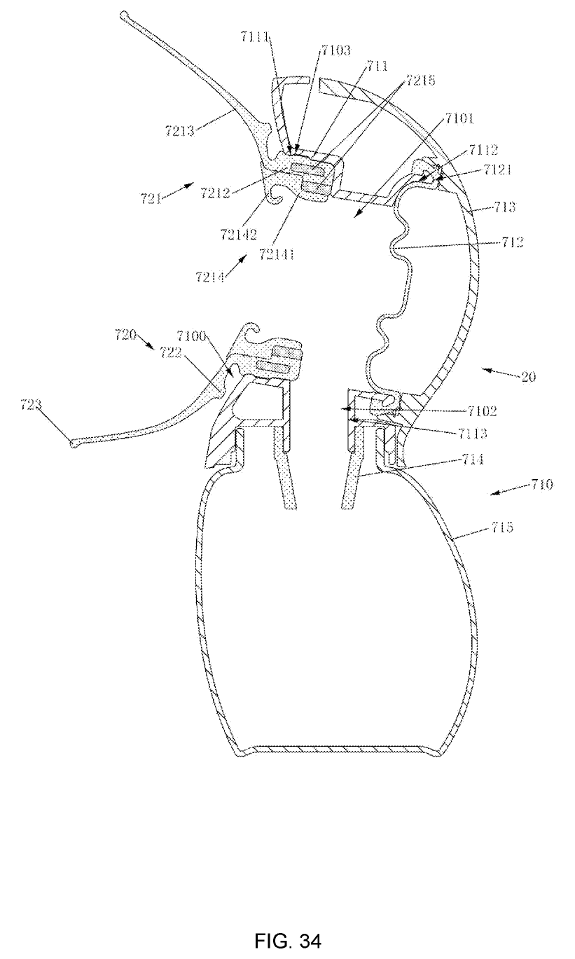

FIG. 34 shows a modified example of the breast pump according to the present invention. Unlike the breast pumps shown in FIGS. 27 to 30, in this specific example of the breast pumping apparatus 20 shown in FIG. 34, the self-adhesive cover 721 comprises the mounting ring 7212 and the horn cover element 7213. The mounting ring 7212 is extended rearward from the horn cover element 7213 and is used to mount the self-adhesive cover 721 to the device body 710. The curvature of the lower inner wall of the horn cover element 7213 is greater than that of the upper inner wall, which makes the shape of the horn cover element 7213 match the shape of the breast, which is beneficial for the horn cover 720 to reliably adhere to the breast.

In addition, in this specific example of the breast pumping apparatus 20, after the self-adhesive cover 721 is installed on the device body 710, the active space 7100 is formed between the horn cover element 7213 of the self-adhesive cover 721 and the device body 710. The support ring 722 is protrudingly disposed on the horn cover element 7213. The support ring 722 supports the self-adhesive cover 721 on the lower side of the self-adhesive cover 721. Thus, during the breast pumping process, the device body 710 will not sag due to its own weight, causing the nipple to lose balance in the milk suction chamber 7101 of the device body 710. This prevents milk leakage and ensures the adhesion of the horn cover 720 and the reliable adhesion of the breast pump to the breast.

Similar to the breast pump shown in FIG. 33, in this specific example of the breast pumping apparatus 20 shown in FIG. 34, the horn cover 720 comprises the tapered portion 723 which is disposed at the periphery of the self-adhesive cover 721 at the external opening of the self-adhesive cover 721. The thickness of the tapered portion 723 is greater than the thickness of the periphery of the self-adhesive cover 721 at the external opening. That is, the tapered portion 723 is disposed at the periphery of the horn cover element 7213 on the side opposite to the mounting ring 7212, and the thickness of the tapered portion 723 is greater than the thickness of the periphery of the horn cover element 7213 on the side opposite to the mounting ring 7212. In this way, the tapered portion 723 can prevent the periphery of the horn cover element 7213 from cracking due to its thinness.

Those skilled in the art should understand that the embodiments of the present invention described above and shown in the accompanying drawings are merely examples and do not limit the present invention. The objectives of the present invention have been fully and effectively achieved. The functions and structural principles of the present invention have been demonstrated and explained in the embodiments, and any variations or modifications may be made to the implementation of the present invention without departing from the stated principles.

What is claimed is:

1. A horn cover for a breast pumping apparatus, having a cover space and comprising an abutting portion located in the cover space, the abutting portion is used to abut against an areola, wherein a surface of the horn cover defining the cover space is a concave arc surface, and a portion of the horn cover defining the cover space is capable of being flipped backward.

2. The horn cover according to claim 1, wherein a top of the horn cover is formed with a top protrusion.

3. The horn cover according to claim 1, wherein a top of the horn cover is formed with a top protrusion, a bottom thereof is formed with a bottom protrusion, and two opposite lateral sides are respectively formed with contraction portions, wherein a vertical distance between the top protrusion and the abutting portion is greater than a vertical distance between the bottom protrusion and the abutting portion, and the vertical distance between the bottom protrusion and the abutting portion is greater than a vertical distance between each contraction portion and the abutting portion.

4. The horn cover according to claim 1, wherein a curvature of a bottom portion of the surface of the horn cover that defines the cover space is greater than a curvature of the top portion thereof.

5. The horn cover according to claim 1, wherein the horn cover comprises a support ring that is protruded from an outer surface of the horn cover.

6. The horn cover according to claim 1, wherein the horn cover comprises a horn portion and a mounting portion extended rearward from a rear of the horn portion, wherein the cover space of the horn cover is formed in the horn portion, and the abutting portion of the horn cover is formed at a connection position between the horn portion and the mounting portion.

7. The horn cover according to claim 6, wherein the mounting portion is extended outward from the rear of the horn portion in a manner that an inner wall of the mounting portion is an inclined inner wall.

8. The horn cover according to claim 6, wherein the horn cover comprises a tapered portion disposed on an outer edge of the horn portion, and a thickness of the tapered portion is greater than a thickness of a periphery of an opening of the horn portion.

9. The horn cover according to claim 6, wherein the horn cover comprises an annular frame disposed on the mounting portion.

10. The horn cover according to claim 6, wherein a hardness of the mounting portion is greater than a hardness of the horn portion.

11. The horn cover according to claim 1, wherein the horn cover comprises a positioning boss or has a positioning groove.

12. The horn cover according to claim 1, wherein the breast pumping apparatus comprises a device body, a rear cover and a milk bottle, and has a negative pressure chamber, wherein the device body comprises:
a cylindrical body;
a deformable portion which is integrally formed at a rear of the cylindrical body, and the device body has a milk suction chamber defined between the cylindrical body and the deformable portion; and
an abutment portion which is integrally formed at a bottom of the cylindrical body, and the device body has a milk discharge channel which is extended from an inner wall of the cylindrical body to an abutment surface of the abutment portion;
wherein the horn cover is provided on a front of a cylindrical body, wherein the rear cover is installed on the cylindrical body, the negative pressure chamber is formed between the rear cover and the deformable portion, wherein the milk bottle is detachably installed on the rear cover.

13. The horn cover according to claim 12, wherein when the milk bottle is installed on the rear cover, a mouth of the milk bottle abuts against the abutment surface of the abutment portion, and the milk discharge channel is capable of connecting the milk suction chamber and a milk storage chamber of the milk bottle.

14. The horn cover according to claim 12, wherein an inner wall of the device body for forming the milk suction chamber is inclinedly extended along a direction from a connection position between the horn cover and the cylindrical body towards the deformable portion.

15. The horn cover according to claim 12, wherein the device body comprises a panel portion integrally formed on the cylindrical body and surrounding a middle of the cylindrical body, wherein an edge of the rear cover is mounted on an edge of the panel portion.

16. The horn cover according to claim 12, wherein the rear cover comprises a cover shell, an inner abutment ring arm, and an assembly ring, wherein the inner abutment ring arm is extended integrally from a middle of the cover shell, and the assembly ring is integrally formed at a bottom of the cover shell, wherein the inner abutment ring arm sealably abuts against the cylindrical body to form the negative pressure chamber between the rear cover and the deformable portion, the assembly ring is located below the abutment portion, and the milk discharge channel corresponds to a ring channel of the assembly ring, an edge of the cover shell is mounted to the edge of the panel portion, the mouth of the milk bottle passes through the ring channel of the assembly ring and is mounted on the assembly ring, and the mouth of the milk bottle abuts against the abutment surface of the abutment portion.

17. The horn cover according to claim 16, wherein the rear cover comprises an outer abutment ring arm that is integrally extended from the middle of the cover shell and is located outside the inner abutment ring arm, wherein the outer abutment ring arm is mounted on an inner side of the panel portion.

18. The horn cover according to claim 12, wherein the breast pumping apparatus comprises a duckbill valve which is disposed on the device body and is used to control connectivity of the milk discharge channel, wherein the duckbill valve is installed on the abutment portion of the device body, wherein the device body and the duckbill valve are integrally made of silicone material.

19. The horn cover according to claim 12, wherein the abutment portion has a vent hole extended from the abutment surface to a side of the abutment portion opposite to the abutment surface.

20. The horn cover according to claim 19, wherein the abutment portion has an exhaust groove being disposed on the side of the abutment portion opposite to the abutment surface, and the exhaust groove is extended from the vent hole to an edge of the abutment portion.
